(19) United States
(12) Patent Application Publication
(10) Pub. No.: US 2010/0211172 A1
(43) Pub. Date: Aug. 19, 2010

(54) Implantable Device For Communicating With Biological Tissue

(75) Inventors: Ravi Bellamkonda, Marietta, GA (US); Dinal Andreasen, Marietta, GA (US); Isaac Clements, Atlanta, GA (US); Young-Tae Kim, Atlanta, GA (US)

Correspondence Address: FISH & RICHARDSON P.C., P.O BOX 1022, Minneapolis, MN, 55440-1022, US

(73) Assignee: GEORGIA TECH RESEARCH CORPORATION, Atlanta, GA (US)

(21) Appl. No.: 12/593,723

(22) PCT Filed: Apr. 2, 2008
(86) PCT No.: PCT/US08/59157

§ 371 Date: Mar. 26, 2010

Related U.S. Application Data
(60) Provisional application No. 60/909,571, filed on Apr. 2, 2007.

(51) Int. Cl. A61F 2/02; A61N 1/00

(52) U.S. Cl. 623/11.11; 607/116

(57) ABSTRACT

Provided are implantable devices for communicating with biological tissue and methods and systems for using the devices. For example, the devices are implanted in a subject and used to communicate with regenerated neural tissue.

### CROSS REFERENCE TO RELATED APPLICATIONS

This application claims the benefit of U.S. Provisional Application No. 60/909,571, filed Apr. 2, 2007, which is incorporated by reference in its entirety as part of this application.

### STATEMENT REGARDING FEDERALLY SPONSORED RESEARCH

This invention was made with government support under Grant DGE-0333411 awarded by the National Science Foundation. The government has certain rights in the invention.

### BACKGROUND

It is often desirable to access tissues of an organism that have the capacity for growth, including regeneration. Such access can enable the influencing, monitoring or measuring of these tissue or other tissues of interest in the organism.

### SUMMARY

Provided are implantable devices for communicating with biological tissue and methods and systems for using the devices. For example, the devices are implanted in a subject and used to communicate with regenerating or growing neural tissue.

### DESCRIPTION OF DRAWINGS

FIG. 1A is s schematic illustration showing a perspective view of an example implantable device.

FIG. 1B is a schematic illustration showing an example electrospinning process for producing a substrate.

FIG. 2A is a schematic illustration showing a perspective view of an example substrate, interfacing unit and tissue grown or regenerated along the substrate surface and interfacing unit.

FIG. 2B is a schematic illustration showing a perspective view of an example substrate, interfacing unit and tissue grown or regenerated along the substrate surface and interfacing unit.

FIG. 3A is a schematic illustration of a perspective view of an example implantable device in communication with a peripheral neural tissue stump and distal neural tissue stump.

FIG. 3B is a schematic illustration of an example electrode array.

FIG. 3C is a magnified view of a portion of the electrode array illustrated in FIG. 3B.

FIGS. 4A-E are schematic illustrations showing perspective views of exemplified uses of an implantable device shown in FIG. 1.

FIGS. 5A and B are schematic illustrations showing perspective views of example multi substrate layer implantable devices.

FIG. 5C is a photograph showing an example electrode pattern of an example interfacing unit.

FIG. 6 is a schematic illustration showing a perspective view of an example implantable device in communication with a wireless transmitter device.

FIG. 7 is a schematic illustration showing an example prosthetic system using an example implantable device.

FIG. 8 is a representation in block diagram form of an example system for use with an example implantable device.

FIG. 9 is a representation in block diagram form of an example system for use with an example implantable device.

FIGS. 10A and B show staining of a dorsal root ganglia (DRG) seeded on a substrate near an overlaid polyimide surface.

FIGS. 11A-C show staining of a longitudinal cross section of a example implantable device showing nerve regeneration along an example substrate and across an example electrode array.

FIGS. 12A-D show a higher magnification view of a portion of the polyimide electrode surface and overlying regeneration shown in FIGS. 11A-C.

FIGS. 13A-F show staining to characterize the inflammatory around an example polyimide electrode.

FIG. 14 shows a stained longitudinal cross section of an implantable device with an isolated nerve segment sutured to the distal end of the implantable device.

FIGS. 15 A and B show electrical activity recorded from an example thin-film electrode in vitro.

### DETAILED DESCRIPTION

Provided are implantable devices for communicating with biological tissue. The devices can have a tissue regenerative/guidance function and an interfacing function. The regenerative function allows for guidance of growing tissue. As used throughout this application, growing tissue includes regenerating tissue. Once growth or regeneration is complete, the implantable device still provides the interfacing function to tissues that have grown or regenerated.

A substrate can be used to provide guidance to the regenerating or growing tissue. Thus, an example implantable device can comprise a substrate having a surface. The substrate can comprise a plurality of uniaxially oriented fibers made of at least one synthetic or natural polymer. Optionally, the fibers can be electrospun, but can be made by other methods. The fibers can also be submicron fibers, including, e.g., nanofibers. Optionally, the fibers are electrospun nanofibers. Optionally, the substrate has a longitudinal axis and the surface is substantially planar. The substrate can be up to about 200 μm thick, e.g., about 1 μm to about 200 μm thick or any amount in between.

The substrate is configured to topographically direct tissue growth along its surface. The term along is not intended to mean that tissue must be in direct contact with the substrate surface itself. Thus, tissue can grow along the substrate surface by growing over cells or other compositions located directly on the surface of the substrate. For example, a substrate surface implanted in a subject may become covered with cells, such as fibroblasts, or a cell layer, such a layer of fibroblasts. Regenerating neural tissue growing across or along covering cells is considered to be growing along the substrate surface, even if there is no direct contact between the substrate surface and the regenerating tissue. Topographically directed means that the substrate provides topographical cues to biological tissue growing along its surface by using physical cues or other spatially distributed biochemical cues to direct the tissue growth. The physical cues and spatially distributed biochemical cues can distribute neural fibers, e.g., in a substantially planar distribution to allow spatial discrimination for specificity in stimulation and/or recordation from individual neural fibers or subsets of neural fibers. The directed distribution can also allow for isolation of the spatially distributed tissue in one or more chambers or other isolating means. Such isolation can be used to provide further specificity and discrimination while reducing background interference.

The implantable device can further comprise an interfacing unit positioned relative to the substrate surface such that tissue grown along the surface is topographically directed into operative communication with the interfacing unit. In one example, the interfacing unit is an electrode. The substrate can support and guide growth of regenerating or growing tissue across the electrode and can keep tissue viable for extended periods of time. The tissue grown along the substrate surface can be neural tissue. Neural tissue growth and continued viability can be facilitated by tropic or trophic guidance and support, which can come from a variety of sources, including glial cells (e.g. Schwann cells or astrocytes), fibroblasts, neurotrophins and muscle targets, or exogenously added sources or compounds.

The interfacing unit is not limited to an electrode. The interfacing unit can be any unit or device configured to interact with tissue in operative communication with the interfacing unit. Interactions can include stimulatory, sensing or monitoring interactions. Thus, an interacting interfacing unit can provide a stimulus to tissue that it is in operative communication with, or the interfacing unit can sense and monitor activity, the environment, or status of the tissue that is in operative communication with the interfacing unit. Examples of interfacing units that can interact with tissue include optical sensors, optical transmitters, chemical sensors, chemical transmitters, mechanical sensors, mechanical stimulators, thermal sensors, thermal transmitters, light transmitters, light receivers, magnetic transmitters, magnetic receivers, fluid transmitters, and fluid receivers as well as electrodes with electrical transmission and receiving properties.

The term operative communication as used herein means that the interfacing unit can interact or communicate with the tissue in accordance with that interfacing unit's functional input or output. For example, if the interfacing unit is an electrode, operative communication means that the electrode can provide an electrical signal output to the tissue or can receive electrical signal input from the tissue. Similarly, a chemical sensor is in operative communication with tissue when it can sense chemicals in or from the tissue. A chemical transmitter is in operative communication when it can provide chemicals or compositions to the tissue. The term does not necessarily imply direct contact between the interfacing unit and the tissue. For example, as would be known to one skilled in the art, an electrode can interact with tissue that is not in direct contact with the tissue so long as the electrode and the tissue are functionally coupled.

Any tissue capable of topographically guided growth along a surface can be used in the described devices and system. Optionally, the tissue grown along the surface and topographically directed into operative communication with the electrode is neural tissue. The neural tissue can be regenerating or regenerated central or peripheral neural tissue. For example, a transected nerve or portion thereof, can be topographically directed in its growth along the surface of the substrate. More specifically, axons or dendritic processes of neurons can be topographically directed as described herein. Support cells can also be included in the neural tissue. Neural tissue, and particularly peripheral nerves, are used throughout by way of example. Other tissues can be used in similar ways.

When neural tissue is grown along the surface, an electrode can be the interfacing unit. Since neural tissue comprises electrically conductive tissue, an electrode in operative communication with neural tissue can receive electrical signals from the neural tissue. Such received electrical signals can be further processed or recorded using recording or processing units in communication with the electrode.

An electrode in operative communication with the neural tissue can also be used to functionally stimulate the neural tissue. Functional stimulation can comprise stimulating an action potential in an axon, subset of axons, or all of the axons of the neural tissue. Thus, an electrode can be configured to functionally activate neural tissue topographically directed into operative communication with the electrode by causing an action potential in an axon, a subset of axons, or all of the axons of the neural tissue. The same or a second electrode can be configured to receive electrical signals from neural tissue topographically directed into operative communication with the electrode.

For an effective electrical interface with neural tissue, an active area of the electrode can be used, which serves to convert ionic current flow in the tissue into electron flow in the conductor. A bidirectional electrode can be used to provide a low impedance transition to the tissue and a high charge transfer capacity. Lower impedance can translate into lower noise in the neural recording case, and high charge transfer translates into effective stimulation without electrolysis and material migration. The electrode can be small enough to allow selective recording from small groups of neurons, an axon, or bundles or subsets of axons to provide a high density of recording sites.

The neural tissue topographically directed into operative communication with the electrode can be an axon, subset of axons, or all of the axons of the neural tissue grown along the surface of the substrate. The electrode can be used to functionally activate or stimulate the axon, subset of axons, or all of the axons of the neural tissue that is in operative communication with the electrode. The electrode can also receive electrical signals from the axon, subset of axons, or all of the axons of the nerve that is in operative communication with the electrode.

As described above, operative communication does not necessarily imply direct contact with a tissue. For example, an axon, subset of axons, or all of the axons of a neural tissue can be placed in operative communication with an electrode when it is guided to within about 200 μm or less from the electrode. Similarly, an axon, subset of axons, or all of the axons of a neural tissue can be placed in operative communication with an electrode when it is guided to within about 100 μm, 50 μm, 25 μm, 10 μm, 5 μm, 1 μm or less from the electrode. An axon can be separated from an electrode by other cells such as glial cells (e.g. Schwann cells or astrocytes).

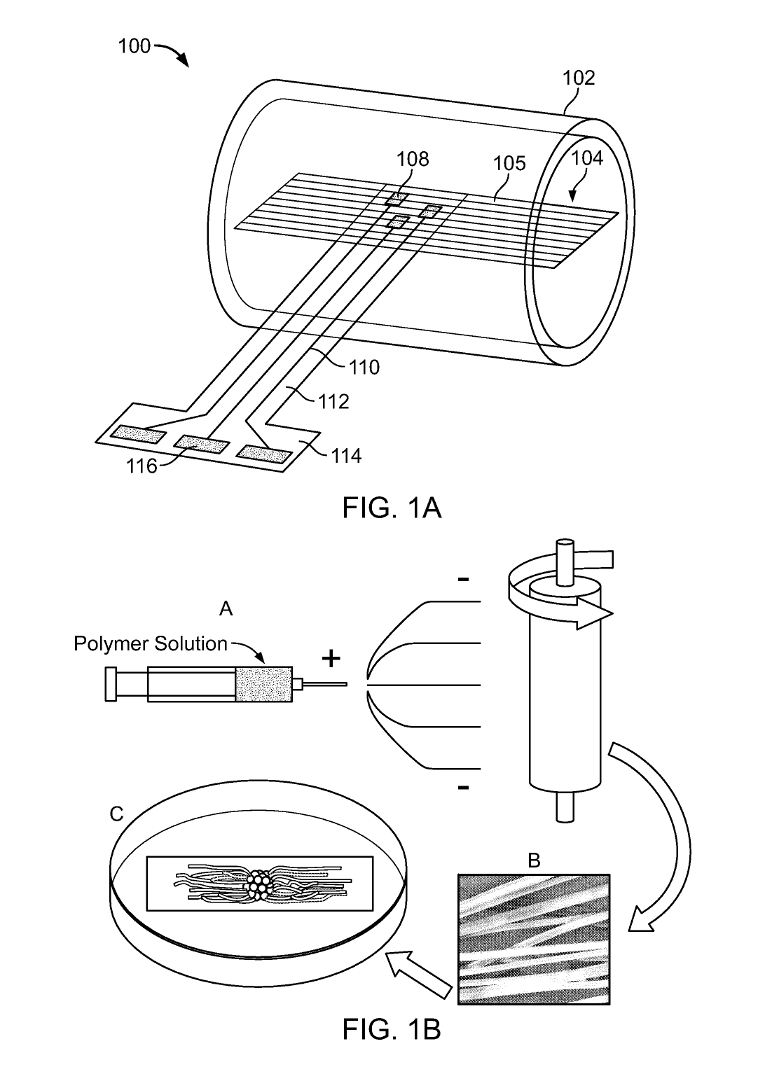

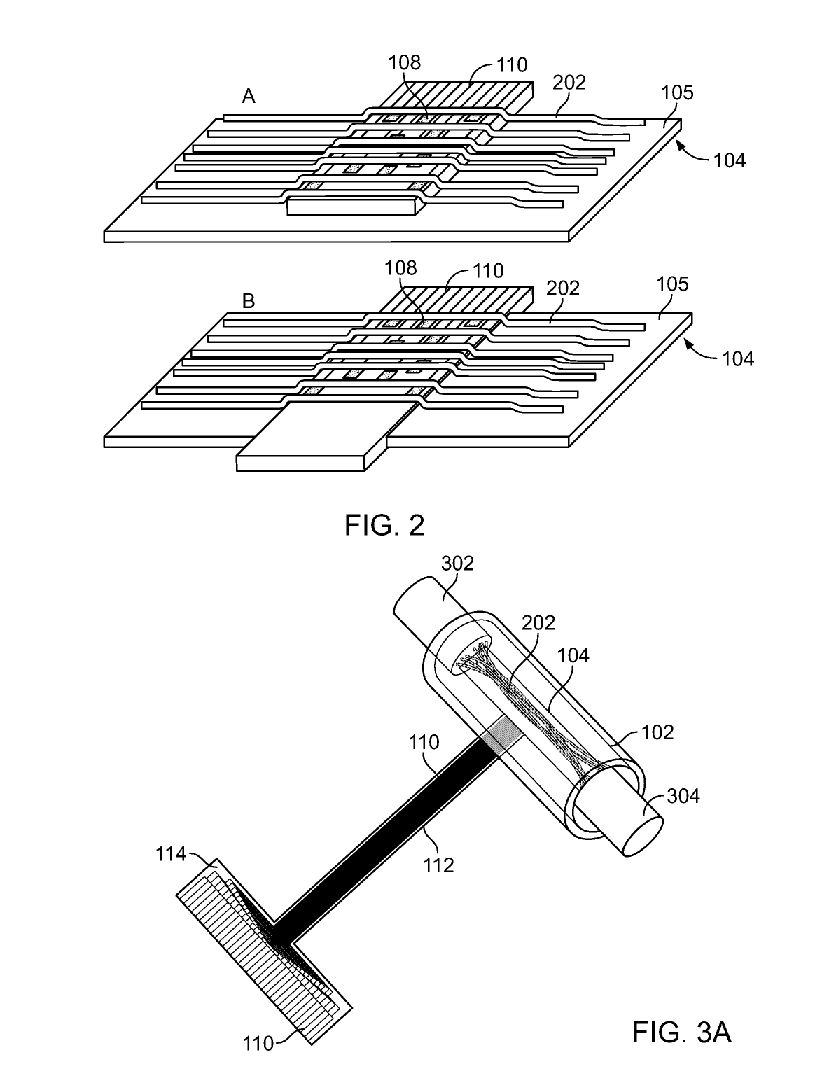

The interfacing unit can be operatively attached in substantial overlying registration with the substrate surface as shown in FIG. 1A. Optionally, the interfacing unit is glued or otherwise secured to the surface of the substrate over which tissue is grown along. In this example, tissue can grow along the surface and over the positions where the interfacing unit is secured to the surface. The interfacing unit can be optionally interposed within the substrate as shown in FIG. 2B.

The substrate itself can be located within a support structure. For example, the substrate can be disposed in a tubular conduit. The substrate can also be disposed in a hydrogel matrix composition in the presence or absence of a tubular conduit. The substrate can be positionally fixed relative to the tubular conduit, hyrodgel matrix or other support structure. Positionally fixing the substrate relative to the support structure can also fix the interfacing unit relative to the support structure as well as the substrate. Thus, an example implantable device can comprise a substrate comprising a plurality of uniaxially oriented electrospun fibers made of at least one synthetic or natural polymer and a support structure to which the substrate is attached, wherein the substrate is positionally fixed relative to the support structure.

Tubular guidance channels can be used to facilitate directed neural growth by isolating regenerating or growing axons from scar tissue and guiding axonal fibers toward their distal targets. Even in the absence of any specific distal target (blind-ended case), neural growth is possible through empty semi-permeable guidance tubes. By adding a fragment of neural tissue to the distal end of a guidance tube, growth or regeneration can be increased to match or exceed levels obtained in the presence of a distal stump. Thus, an isolated neural tissue fragment supports axonal regeneration or growth at least as well as the intact distal nerve stump that is still connected to the end organ. For example, a very small segment of neural tissue is more than sufficient for providing a source of migrating Schwann cells, which support the regeneration of neural tissue across a gap. Mammalian peripheral nerves can be stimulated to regenerate normally after amputation by molecular signaling derived from sources other than the intact distal stump.

After limb amputation, a portion of the nerve proximal to the incision site remains viable. The mechanism by which axotomized spinal motor neurons are able to survive in the absence of a distal target lies in the neurotrophic support they receive from local sources, especially Schwann cells. Schwann cells migrate after injury and differentiate to provide mechanical and neurotrophic support to injured axons. Schwann cells are capable of remaining in the proximal nerve stump indefinitely, providing a substitute trophic target for axons lacking their original targets. Neurons grow stable, and they retain their ability to conduct action potentials. Human amputee experiments have demonstrated the practical potential of the long-term viability of severed peripheral nerves. A high percentage of both afferent and efferent fibers survive a small distance back from the site of initial trauma, even in the absence of their original distal targets.

The substrate, for example, one comprising oriented fibers such as electrospun fibers, nanofibers, or electrospun nanofibers, can be used to control the physical location of regenerated or growing axons, thus allowing for directed neural growth across embedded thin-film electrodes. Thin-film fabrication techniques can provide up to 64 channels or more on each electrode. Multiple electrodes can also be integrated into stacks of electrospun films, allowing for hundreds of channels with appropriate multiplexing. Wireless technology can also be integrated into the implantable device to allow for non-invasive access to signals. A non-degradable polyacrylonitrile-methacrylate (PAN-MA) polymer based electrospun film can be used, which can preserve the location of the growing or regenerated neural tissue, preventing axonal remodeling that might result with the use of degradable polymer substrates. However, both the electrospinning technique, as well as the integration of electrodes can be used with both degradable and non-degradable polymers.

The aligned nanofibers or electrospun fibers can be fabricated from PAN-MA using an electrospinning process. PAN-MA films have been used in dialysis tube and macroencapsulation applications. PAN-MA films of different diameters (e.g., sub 200 nm, 300-500 nm, or 800-1000 nm), can be generated using different concentrations of polymer solution. The thickness of each film can be dictated by the duration of electrospinning Fiber diameter and orientation can be evaluated using scanning electron microscopy.

Two example types of electrodes that can be used include a commercially available (MultiChannel Systems, Reutlingen, Del.) thirty-two channel thin-film electrode. A second type can be a custom fabricated iridium oxide electrode, featuring sixty-four recording sites. The thin-film electrodes can be integrated into the electrospun films. For example, an electrode can be overlaid and glued on top of the electrospun film. In another example, the electrode can be interposed between two sections of the film such that it is level with the film surfaces.

Regenerating axons from a fully transected or partially cut nerve (e.g., epineural window) can be encouraged to grow along the integrated thin-film electrode array/substrate surface to establish one or more interfacing or communication sites. The electrode can be integrated within the tube or matrix and can take up minimal area in the plane normal to the direction of regeneration. The term tube is not limited to a lumen having a circular cross section. Thus a tube can have any cross sectional shape provided it functionally supports the substrate and integrated electrode and allows for tissue to grow or regenerate along the substrate. As a result, neural tissue growth or regeneration is not impeded by the substrate or integrated array. For example, in the case of neural tissue regenerating or growing through a tubular conduit with a diameter of 1.6 mm, the transverse cross-sectional area within the conduit available for regeneration is 2.01 mm². A planar thin-film electrode of 12 μm thickness integrated into the conduit, such that the plane of the electrode surface bisects the top and bottom longitudinal halves of the conduit, occupies about 1% of the transverse cross-sectional area. Multiple electrode arrays can be integrated with stacked layers of substrate, which also take up minimal area in the plane normal to the direction of regeneration.

Minimizing the blocked area within a guidance tube or scaffold in the plane normal to the direction of regeneration can be desirable to minimize interference with the progress of growing or regenerating tissues. Minimizing interference with the progress of regenerating neural tissue, for example, can lead to healthier patterns of regeneration resulting in more numerous, larger, more viable regenerated axons. Minimizing interference with the progress of regenerating tissues can also allow these tissues to continue regenerating through the implantable device and towards natural or artificially provided distal targets. These distal targets can provide trophic support to regenerated axons, increasing their long-term viability. Also, natural distal targets can be innervated or reinnervated by regenerating axons, putting the distal target back under neural control. This situation can be useful to enable functional electrical stimulation of the distal target via integrated electrode(s). Exogenous supporting cells, tissues, or growth factors can be seeded within the implantable device. Growth factors can be provided in a sustained release form.

If the integrated substrate electrode is disposed with in a tube lumen, the blocked transverse cross sectional area or the area within the tube normal to the direction of regeneration by the substrate/electrode can be about 70% or less. For example, the blocked lumen area can be about 60%, 50%, 40%, 30%, 20%, 10%, 5%, 1% or values in between the recited values. Optionally, when using one substrate layer, about 1.5% or less of the area within the tube is blocked in a tube of about 1.6 mm. In larger diameter tubes, this percentage can be lower since the thickness of the substrate and interfacing unit can remain constant.

One example of an implantable device for communicating with biological tissue is configured for communicating with a nerve. The device can comprise a substrate having a surface as described above. The substrate can topographically direct growth of the nerve or a portion thereof along its surface. The device can further comprise a first electrode positioned relative to the substrate surface such that the nerve or portion thereof grown along the surface is directed topographically into operative communication with the first electrode.

The first electrode can stimulate an action potential in an axon, a subset of axons, or all axons of the nerve or portion thereof when in operative communication with the nerve or a portion thereof. The first electrode can also receive electrical signals from an axon, a subset of axons, or all of the axons of the nerve when in operative communication with the nerve or a portion thereof. The implantable device can also comprise a second electrode positioned relative to the substrate surface such that the nerve or portion thereof grown along the surface is topographically directed into operative communication with the second electrode. The second electrode can receive electrical signals from the nerve or a portion thereof. Optionally, the second electrode can stimulate an action potential in an axon, subset of axons, or all axons of the nerve when in operative communication with the nerve or a portion thereof.

Another example of an implantable device for communicating with biological tissue such as neural tissue comprises a substrate having a surface, wherein the substrate is configured to topographically direct biological tissue growth along its surface. The device can further comprise a plurality of electrodes positioned relative to the substrate surface such that tissue grown along the surface is directed into operative communication with one or more of the electrodes. The plurality of interfacing units can also include optical sensors, optical transmitters, chemical sensors, chemical transmitters, mechanical sensors, mechanical stimulators, thermal sensors, thermal transmitters, light transmitters, light receivers, magnetic transmitters, magnetic receivers, fluid transmitters, and fluid receivers, electrodes and combinations thereof.

The implantable devices described herein can be functionally integrated into systems for interacting with a target. An example system for activating a target can comprise an implantable substrate having a surface. The substrate is configured to topographically direct neural growth or regeneration along its surface. The system can further comprise an electrode positioned relative to the substrate surface such that neural tissue grown along the surface is topographically directed into operative communication with the electrode. The electrode can be configured to receive electrical signals from an axon, a subset of axons, or all axons of the neural tissue when in operative communication with the neural tissue. The system can further comprise a processing unit for determining a pattern of received electrical signals and for activating a target based on the determined pattern. The target can include a prosthetic device, a computer or an organ. The processing unit can be an external or internal processing unit.

The implantable devices described herein can be also be functionally integrated into a prosthetic system. An example prosthetic system can comprise an implantable substrate having a surface. The substrate is configured to topographically direct neural tissue growth along its surface. The prosthetic system further comprises a plurality of electrodes positioned relative to the substrate surface such that neural tissue grown along the surface is topographically directed into operative communication with the electrodes.

One or more electrode of the plurality is configured to stimulate an action potential in an axon, subset of axons, or all axons of the neural tissue and one or more electrodes of the plurality is configured to receive electrical signals from an axon, subset of axons, or all of the axons of the neural tissue. The same electrodes configured to stimulate an action potential, or different electrodes, can also be configured to receive electrical signals. The system further comprises a prosthetic device having a sensor, an actuator and a processor unit configured for activating an electrode to stimulate an action potential in an axon, subset of axons, or all axons of a neural tissue when the sensor of the prosthetic device has been activated and to activate the actuator of the prosthetic device when an electrode has received an electrical signal from an axon, a subset of axons, or all of the axons of a neural tissue. A peripheral neural tissue interface for amputees can have one or more of the following characteristics: a) can be used ‘off-the-shelf’ and is easily implantable in amputees; b) facilitates neural tissue interactions with many electrode sites for the establishment of high resolution recording and stimulation capabilities; c) connections remain stable over long periods of time.

The devices can be used for implantation into human and veterinary subjects. For example, in cases of nerve injury, an implantable device can be implanted in a subject to interface with a proximal nerve stump. The implantable device is also useful in an amputee to provide closed-loop control of a prosthetic limb. The nerve stump, in either case, can be sutured into the implantable device and allowed to grow for several weeks to months. Axons grow along the substrate surface and into operative communication with one or more interfacing units. The electrodes can detect motor commands that previously controlled the amputated arm. These detected signals can be transmitted percutaneously, wirelessly or otherwise to the prosthetic limb.

The implantable device can also receive artificial sensory input from the prosthetic limb. Electrodes in operative communication with sensory axons in the subject can be stimulated to recreate the lost sensations that previously accompanied the natural limb. The devices can also be used for functional electrostimulation. For example, a nerve can be transected or accessed using end-to-side techniques. The device can then be used for functional electrostimulation of nerves innervating muscles. Functional electrostimulation can be used to restore motor function to paralyzed individuals, for example. The devices can also be used to augment nerve regeneration by applying electrical stimulation to regenerating axons and can be used in research applications to elucidate the function of peripheral nerves and to determine changes in their electrophysiological and functional properties.

FIG. 1 shows an example implantable device 100 for communicating with biological tissue. The example implantable device can comprise a substrate 104 having a surface 105. The substrate is configured to topographically direct tissue growth along the surface 105. Topographically directed means that the surface or substrate provides topographical cues to biological tissue growing along the surface 105 by means of physical cues or other spatially distributed biochemical cues to direct the tissue growth across the surface 105.

The implantable device can further comprise an interfacing unit. The interfacing unit can be positioned relative to the substrate surface 105 such that tissue grown along the surface is topographically directed into operative communication with the interfacing unit. Optionally, the tissue grown along the surface 105 can be directed into operative communication with an interfacing unit that comprises one or more electrodes 108.

Electrodes can be integrated into a flex circuit 112 wherein the electrodes are in communication with connector pads 116 through conducting wires or traces 110. The connector pads 116 can further connect the implantable device to a processor system where other monitoring or control functions can be directed. Thus, 116 represents a functional connection point to the next level of circuitry to provide an integrated system.

The substrate can topographically guide or direct a regenerating or growing tissue of interest along its surface in a direction of regeneration or growth to a locality where that tissue can be interrogated or communicated with using the interfacing units. For example, the electrical signals can be received from the tissue or electrical stimulation can be supplied to the tissue. Other types of non-electrode interfacing units can also be used. For example, an interfacing unit can be selected from the group consisting of optical sensors, optical transmitters, chemical sensors, chemical transmitters, mechanical sensors, mechanical stimulators, thermal sensors, thermal transmitters, light transmitters, light receivers, magnetic transmitters, magnetic receivers, fluid transmitters, and fluid receivers electrodes and combinations thereof.

The substrate can comprise a plurality of uniaxillary oriented fibers made from at least one synthetic or natural polymer. Optionally, the fibers used in the substrate can have a diameter from about 40 nm to about 1500 nm. For example, the fibers can have a diameter from about 200 nm to about 1000 nm, or from about 400 nm to about 1000 nm. In one example, the fibers have a diameter between 500 and 800 nm. The fibers can be produced by electrospinning techniques or other techniques.

The uniaxial oriented fibers can have greater than 50% of the fibers oriented within 40° of an axis, i.e., +/−20° of the axis. The fibers can be oriented in the implantable device over several millimeters in length, e.g., between 2 and 100 mm. Optionally, at least 60%, at least 75%, or at least 85% of the fibers are within 20 degrees of the uniaxial orientation.

The implantable device can be used in vivo, i.e., by implantation into a subject in need of tissue regeneration or growth, such as at an injury (or disease) site, to heal neural, cartilage, bone, cardiovascular and/or other tissues. Optionally, the implantable device is used in the regeneration of tissues of the peripheral nervous system or the central nervous system. For example, the implantable device can be implanted into an injured sciatic or cavernous nerve, or into a spinal cord or brain site.

The fibers can be formed from at least one polymer, e.g., a synthetic polymer. Optionally, the polymer is a biocompatible, thermoplastic polymer. Examples of which are known in the art. Optionally, the polymer is a polyester or polyamide suitable for use in in vivo applications in humans. The polymer can be biodegradable or non-biodegradable, or can include a mixture of biodegradable and non-biodegradable polymers. Representative examples of synthetic polymers include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polyalkylenes such as polyethylene and polypropylene, polyalkylene glycols such as poly(ethylene glycol), polyalkylene oxides such as poly(ethylene oxide), polyvinyl alcohols, polyvinyl ethers, polyvinyl esters, polyvinylpyrrolidone, poly(vinyl alcohols), poly(butyric acid), poly(valeric acid), and poly(lactide-co-caprolactone), copolymers and blends thereof.

As used herein, derivatives include polymers having substitutions, additions of chemical groups, for example, alkyl, alkylene, hydroxylations, oxidations, and other modifications routinely made by those skilled in the art. Examples of biodegradable polymers include polymers of hydroxy acids such as lactic acid and gly colic acid, and copolymers with polyethylene glycol (PEG), polyanhydrides, poly(ortho)esters, poly(butyric acid), poly(valeric acid), poly(lactide-co-caprolactone), blends and copolymers thereof. Optionally, the biodegradable polymer fibers includes a poly(caprolactone), a poly(lactic-co-glycolic acid), or a combination thereof.

In another example, the non-biodegradable polymer fibers includes a poly (aery lonitrile). Non-degradable polymers can be selected for applications where structural support from the substrate is desired or where elements such as electrodes or microfluidics are incorporated into the substrate.

In another example, the fibers are formed from at least one natural polymer. Examples of suitable natural polymers include proteins such as albumin, collagen, gelatin, Matrigel™ (BD Biosciences, San Jose, Calif.), fibrin, polypeptide or self-assembling peptide based hydrogels, and prolamines, for example, zein, and polysaccharides such as alginate, agarose, cellulose and polyhydroxyalkanoates, for example, polyhydroxybutyrate or any combination thereof.

Optionally, the structure of the implantable device includes multiple, stacked substrate layers, i.e., films, of the uniaxially oriented fibers. In one example, each layer is about 10 μm thick. Thicker or thinner layers can also be used; however, the thickness typically is selected to be one capable of handling and manipulation to stack. For example, the film thickness can enable manual handling, such as to facilitate separation from a (temporary) form on which the fibers are electrospun. Each layer can be oriented such that the fiber orientation in the stack is essentially the same. That is, the axial direction of all layers is pointing in substantially the same direction. Optionally, the stacked structure includes a spacer between some or all of the layers of uniaxially oriented fibers. The spacer can provide sufficient openings to permit cells to infiltrate the substrate and attach to the oriented fibers. The spacer can be water soluble or water insoluble, porous or non-porous. Optionally, the spacer is biocompatible, and can be bioerodible/biodegradable. The spacer can have a thickness between about 25 and about 800 μm. Optionally, each spacer layer in the stack has a thickness of about 50 to about 250 μm. In one example, the spacer includes a hydrogel, such as a thermo-reversible (i.e., temperature responsive) hydrogel. The implantable device can comprise alternating layers of oriented fibers and layers of a hydrogel or other spacer. The hydrogel, for instance, can be an agarose hydrogel or other hydrogel. Examples of which are known in the art. In other examples, the spacer material can be another gel or gel-like material, such as polyethylene glycol, agarose, alginate, polyvinyl alcohol, collagen, Matrigel™ (BD Biosciences, San Jose, Calif.), chitosan, gelatin, or any combination thereof.

The uniaxially aligned fibers provided in the implantable device can be in a form other than a plurality of layers. Optionally, the substrate of the implantable device is the result of rolling one layer, i.e., a film, of aligned fibers in on itself to form a spiral-like roll.

The substrate optionally can be disposed in a secondary structure for containing, positioning, or securing the uniaxially oriented fiber substrate, and/or for further directing tissue growth or regeneration. For example, the secondary structure can be a tubular conduit, in which the substrate/spacer structure can be contained and through which a neural tissue bridge can be grown between two neural stumps. This tube can also made of a biocompatible polymer suitable for use in vivo. The polymer can be biodegradable or non-biodegradable, or a mixture thereof. For example, the secondary structure, for example a tube, can be a polysulfone. The secondary structure can be substantially flexible or rigid, depending upon its particular performance needs.

The fibers can be made by essentially any technique, examples of which are known in the art. Optionally, the fibers are made using an electrospinning technique. Any biocompatible polymer that is amenable to electrospinning can be used. The electrospinning equipment can include a rotating drum or other adaptation at the collector end to generate fibers. The fibers can also optionally be made by micromachining or with masking techniques.

By way of example, the substrate can further include one or more bioactive agents, which can be presented or released to enhance tissue regeneration. As used herein, the term bioactive agent refers a molecule that exerts an effect on a cell or tissue. Representative examples of types of bioactive agents include therapeutics, vitamins, electrolytes, amino acids, peptides, polypeptides, proteins, carbohydrates, lipids, polysaccharides, nucleic acids, nucleotides, polynucleotides, glycoproteins, lipoproteins, glycolipids, glycosaminoglycans, proteoglycans, growth factors, differentiation factors, hormones, neurotransmitters, prostaglandins, immunoglobulins, cytokines, and antigens. Various combination of these molecules can be used. Examples of cytokines include macrophage derived chemokines, macrophage inflammatory proteins, interleukins, tumor necrosis factors. Examples of proteins include fibrous proteins (e.g., collagen, elastin) and adhesion proteins (e.g., actin, fibrin, fibrinogen, fibronectin, vitronectin, laminin, cadherins, selectins, intracellular adhesion molecules, and integrins). In various cases, the bioactive agent can be selected from fibronectin, laminin, thrombospondin, tenascin C, leptin, leukemia inhibitory factors, RGD peptides, anti-TNFs, endostatin, angiostatin, thrombospondin, osteogenic protein-1, bone morphogenic proteins, osteonectin, somatomedin-like peptide, osteocalcin, interferons, and interleukins.

Optionally, the bioactive agent includes a growth factor, differentiation factor, or a combination thereof. As used herein, the term growth factor refers to a bioactive agent that promotes the proliferation of a cell or tissue. Representative examples of growth factors that can be useful include transforming growth factor-α (TGF-α), transforming growth factor-β (TGF-β), platelet-derived growth factors (PDGF), fibroblast growth factors (FGF), nerve growth factors (NGF) including NGF 2.5s, NGF 7.0s and β NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), (e.g., TGFs α, β, β1, β2, and β3), any of the bone morphogenic proteins, skeletal growth factor, bone matrix derived growth factors, and bone derived growth factors and mixtures thereof. As used herein the term differentiation factor refers to a bioactive agent that promotes the differentiation of cells. Representative examples include neurotrophins, colony stimulating factors (CSF), and transforming growth factors. Some growth factors can also promote differentiation of a cell or tissue. Some differentiation factors also can promote the growth or regeneration of a cell or tissue. For example, TGF can promote growth and/or differentiation of cells.

The bioactive agent can be incorporated into the substrate in a variety of different ways. Optionally, the bioactive agent is located and/or formulated for controlled release to affect the cells or tissues in or around the oriented fibers of the substrate. For instance, it can be dispersed in a controlled release matrix material. In one example, the bioactive agent is provided in lipid microtubules or nanoparticles selected to modulate the release kinetics of the bioactive agent. Such particles can be dispersed among the fibers, or provided in or on one or more layers in the substrate. In another example, the bioactive agent is actually integrated into, or forms part of, the fibers themselves. This can be done, for example, by adding the bioactive agent to a polymer solution prior to electrospinning the solution to form the oriented fibers. Release of the bioactive agent can be controlled, at least in part, by selection of the type and amounts of bioerodible or biodegradable matrix materials in the nanoparticles or fibers.

By way of example, the substrate for tissue regeneration includes at least two layers which comprise a plurality of uniaxially oriented, polymeric fibers, wherein at least 75% of the fibers are oriented within 20 degrees of the uniaxial orientation and wherein the layers are stacked and oriented such that the fiber orientation of among the layers is substantially identical; one or more spacers in the stacked layers, between the at least two layers of uniaxially oriented fibers, wherein the spacers comprise a hydrogel.

The implantable devices can be adapted to a variety of tissue growth and regeneration applications, where guided invasion/migration of endogenous or transplanted cells is desired. Different densities of fibers for a given volume of substrate can be used depending, for example, on tissue type. These parameters can be routinely determined for various tissues. The implantable device and systems can be applied to the growth and regeneration of, for example, cartilage, bone, neural, and cardiovascular tissues. In addition, the devices can have other in vivo and ex vivo uses including wound repair, growth of artificial skin, veins, arteries, tendons, ligaments, cartilage, heart valves, organ culture, treatment of burns, and bone grafts.

The interfacing units, for example, an electrode 108, can be integrated with or can be positioned in substantially overlying orientation with the surface 105. For example, electrodes 108 can be glued or otherwise attached to the surface 105 of the substrate 104. The electrodes 108 are designed to allow for tissue growth along the surface 105 so that the tissue grows over and makes operative communication with the electrodes 108. Thus, an axon growing along the surface, for example, can grow over an electrode 108 and back onto the surface 105 thereby growing along the surface.

As described above, the electrodes do not need to physically touch the tissue to be in operative communication. Rather the tissue can be brought within functional proximity to allow such operative communication. For example, for communicating with an axon or with neural tissue, an electrode can be directed to within 200 microns or less of the nerve or axon. However, the distance under which operative communication is possible can depend on the architecture of the electrode and the desired recording or stimulating parameters desired. Similarly, other types of interfacing units can be positioned in relation to the surface to allow for operative communication. Proximity for other interfacing units to be in operative communication with the tissue can be determined.

Two example configurations for regenerating or growing tissue along the surface 105 and an interfacing unit, such as an electrode 108, are shown in FIGS. 2A and B. In FIG. 2A, the example interfacing unit is shown positioned in substantial overlying registration with the surface 105 of the substrate. In this example, nerves or other regenerating or growing tissue 202 are topographically guided along the surface 105 and are not impeded by the interfacing unit. This configuration allows guided growth or regeneration to occur over the interfacing unit and back along the surface for continued growth. As described above, however, cells other than the directed tissue can grow along the interfacing units and surface. The tissue that is being directed over the interfacing units and substrate surface can actually be growing over a cellular layer attached to the interfacing units and substrate surface. Where the regenerating tissue 202 has grown over the interfacing unit, portions of the tissue, including axons, subsets of axons, or all axons or a neural tissue, are brought into operative communication with electrodes 108 or other interfacing units.

FIG. 2B shows an alternative configuration wherein interfacing units are positioned in an integrated position with the substrate 104 such that the interfacing units are substantially in plane with the surface of the substrate 105. This configuration also allows for regenerating or growing tissue such as an axon or axon bundle 202 to grow over an interfacing unit and to make operative communication with the interfacing unit.

In some examples, electrospun fibers or other guidance cues can be positioned on the interfacing units to provide topographical guidance over the interfacing units themselves. For example, grooves on the electrode or sparse fibers on the electrode could provide topographic guidance over the interfacing unit.

In some examples, a plurality of interfacing units are provided for interfacing with tissue such as neural tissue growing along the surface 105. In these cases, afferent and efferent axons or bundles of axons can contact the electrodes. Patterns of electrodes in contact with either afferent or efferent axons, subsets of axons, or all axons of a neural tissue, can be mapped using similar methods as those used for cochlear implants. Once mapped, the implantable device can be used to stimulate desired efferent or afferent axons or can be used to receive signals or information from desired efferent or afferent axons. Such mapping can result in a pattern which allows integrated sensory and motor functionality of a prosthetic device or other target, such as a computer or organ.

The substrate 104 and portions of the implantable device including the integrated interfacing unit can be disposed or located in a support structure. For example, as shown in FIG. 1 the substrate and interfacing units can be disposed in the lumen of a tubular structure 102. The substrate 104 can be positionally fixed relative to the structure 102. In this way, the substrate 104 remains relatively in the same position to the tube 102 throughout implantation and functional use. The support structure 102 can also be shaped other than a tube and can also be a hydrogel matrix or other similar support mechanism. The substrate 104 can be disposed in the tube and fixed positionally within the tube by cutting a slit along a length of the tube wall and positioning the substrate through the slit and closing the slit down over the edge. The slit can be glued in a closed position over the substrate. In one example, a biocompatible UV curing glue can be used to close the slit.

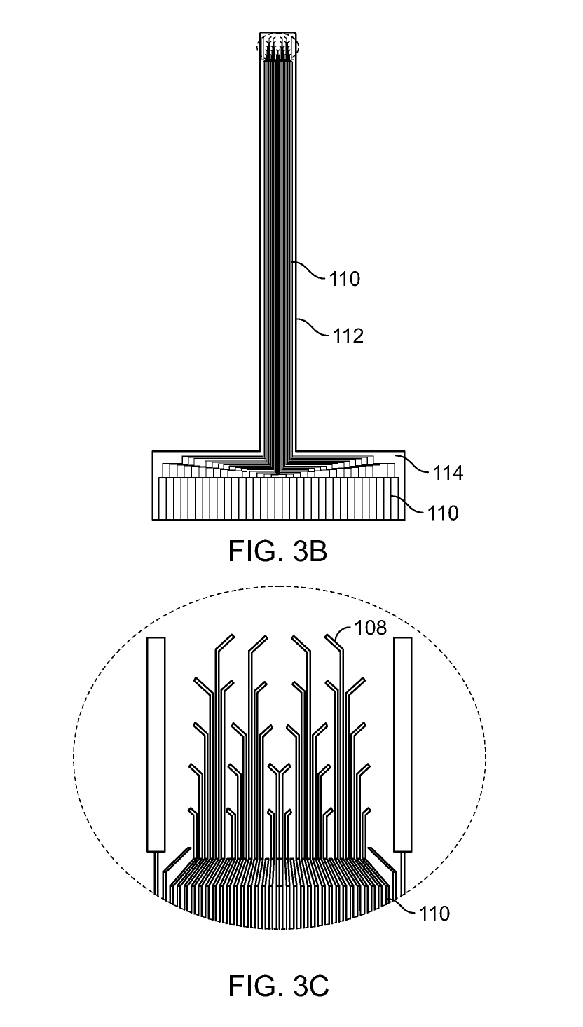

FIG. 3A shows an example use of an implantable device wherein a proximal nerve stump 302 and a distal nerve stump 304 are positioned on a proximal side of the implantable device and on a distal side of the implantable device respectfully. In this example, neural processes from the proximal stump 302 grow along the substrate surface using topographical cues and towards the distal stump 304. Optionally, the growing neural tissue can continue to grow to a target in the body which provides trophic and other support for robust regeneration or growth of the nerve. The neural tissue growing along from the proximal to the distal stump can grow along the surface of the substrate and can be brought into operative communication with an interfacing unit. The interfacing unit can be used to provide stimulation to the neural tissue and to receive sensory information from the neural tissue.

The implantable device can be used to treat a severed nerve where a portion of the injured nerve was removed (e.g. during surgery or injury) and the implantable device is used to regenerate or grow axons along the surface. The natural target can be stimulated using the implantable device and the distal nerve portion stimulated to functionally bypass the proximal portion. The distal stump 304 can also represent a piece of distal neural tissue that is not connected to the target. A distal stump that is not connected to a distal target can provide tropic support for axons growing from the proximal stump along the substrate surface. The substrate does not prevent the growth of axons or neural tissue so that axons or neural tissue can interact with a distal target to provide increased viability of the regenerated axons or dendrites.

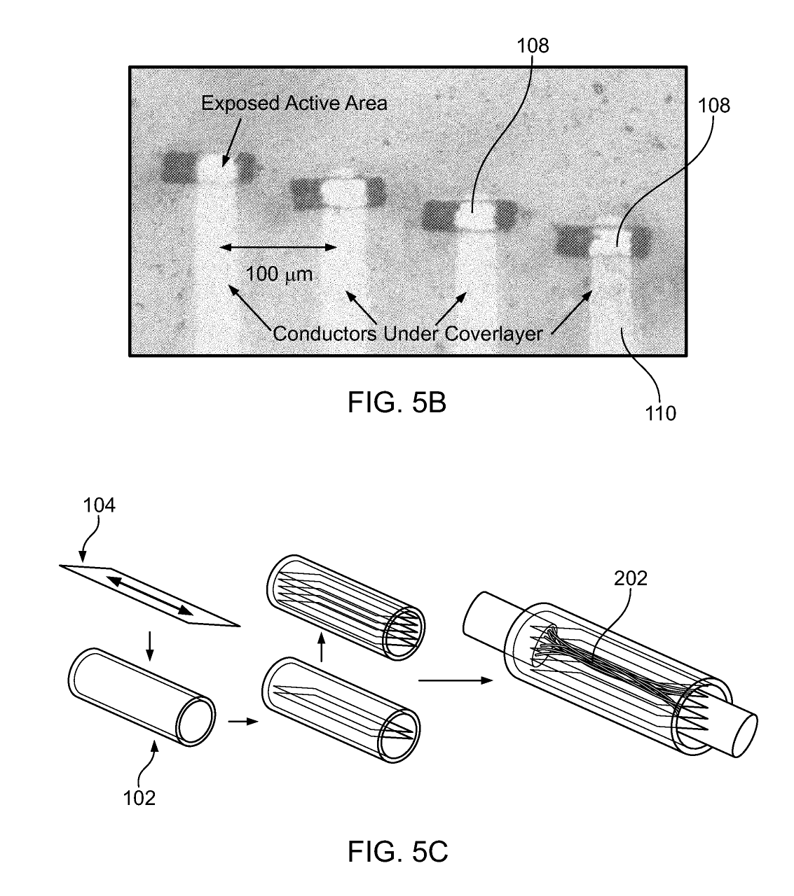

FIGS. 5A and 5B show a pattern of electrodes 108 for positioning on a substrate. The electrodes 108 or other interfacing units can be stepped or otherwise patterned in relation to the surface of the substrate. Any pattern can be used to provide a desired level of surface area coverage of the substrate surface. Optionally a thin-film electrode array can be used. The tissue interfacing portion of the array can include a plurality of individual electrode sites. For example, up to 32 electrodes or more can be used to form the array. The base of the thin film electrode array can be broadened at the non-tissue interfacing end so that connections can be made to access signals from each interfacing electrode site. The thin film electrode array can be manufactured using techniques developed for semiconductor fabrication and modified for the deposition of conductors with 1 μm dimensional tolerance on thin films. The tissue interfacing area of the electrode can comprise a biocompatible material for low impedance electrical connections to biological tissue including gold, iridium oxide, and titanium nitride. The insulating or support portion of the film can be fabricated with polyimide, parylene or silicon, for example.

FIGS. 3B and 3C show another example architecture for the electrodes. The example pattern shown in FIGS. 3B and 3C is a multi-channel or multi-electrode array. The number of electrodes in an array can vary. The number used can be determined based on, for example, the desired resolution or the size of the tissue. A plurality of electrodes or interfacing units can cover a given surface area and make operative communications with a plurality of axons or axon bundles or segments, for example.

Electrodes also do not have to be planar or flat on the surface. For example, the electrodes themselves can have a third dimension, for example, a pyramidal or other shape having a height relative to the surface of the substrate. The architecture of the electrodes can be modified to have lower impedance recordings to strengthen the electrical signal. Three dimensional electrodes can be used to record from axons that may be growing a distance from the surface.

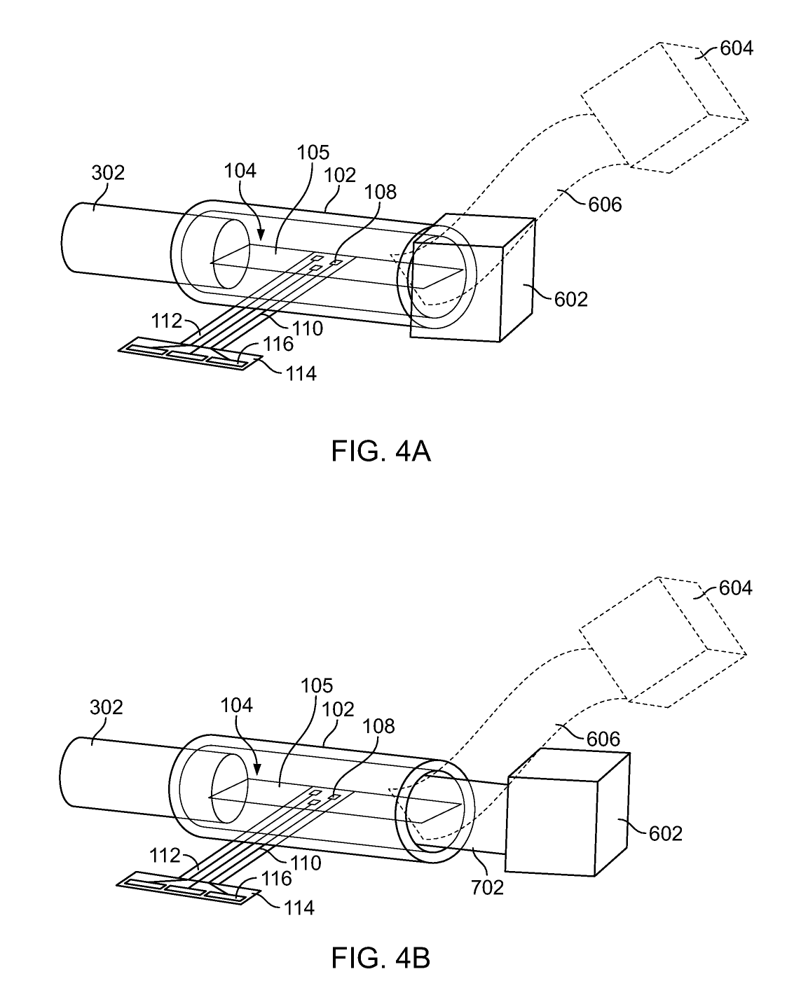

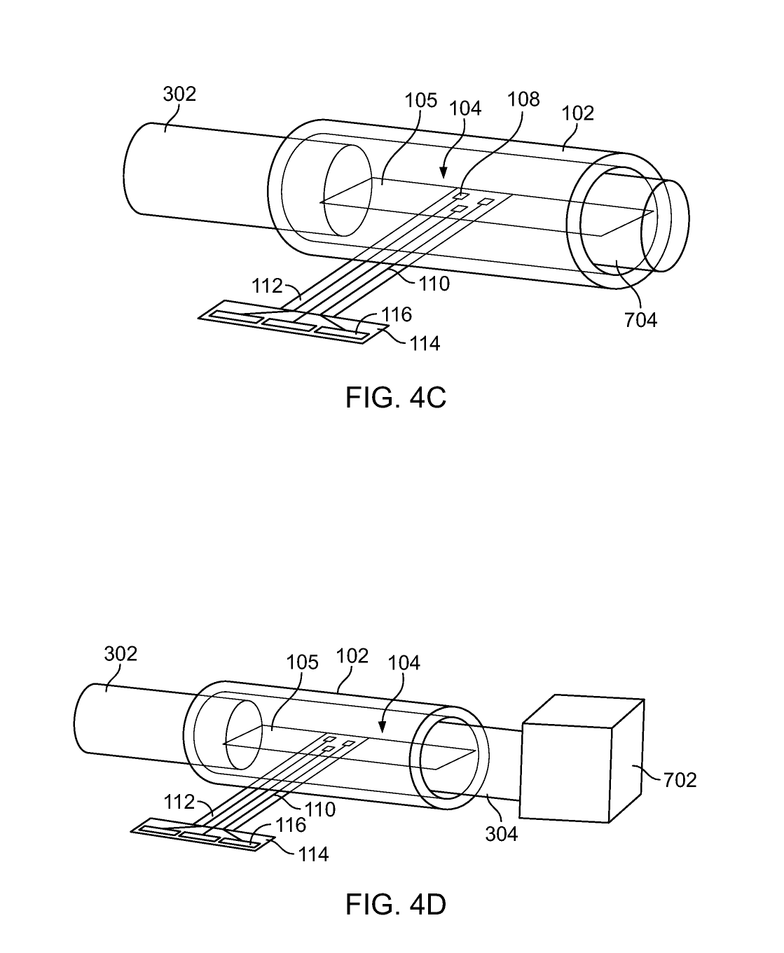

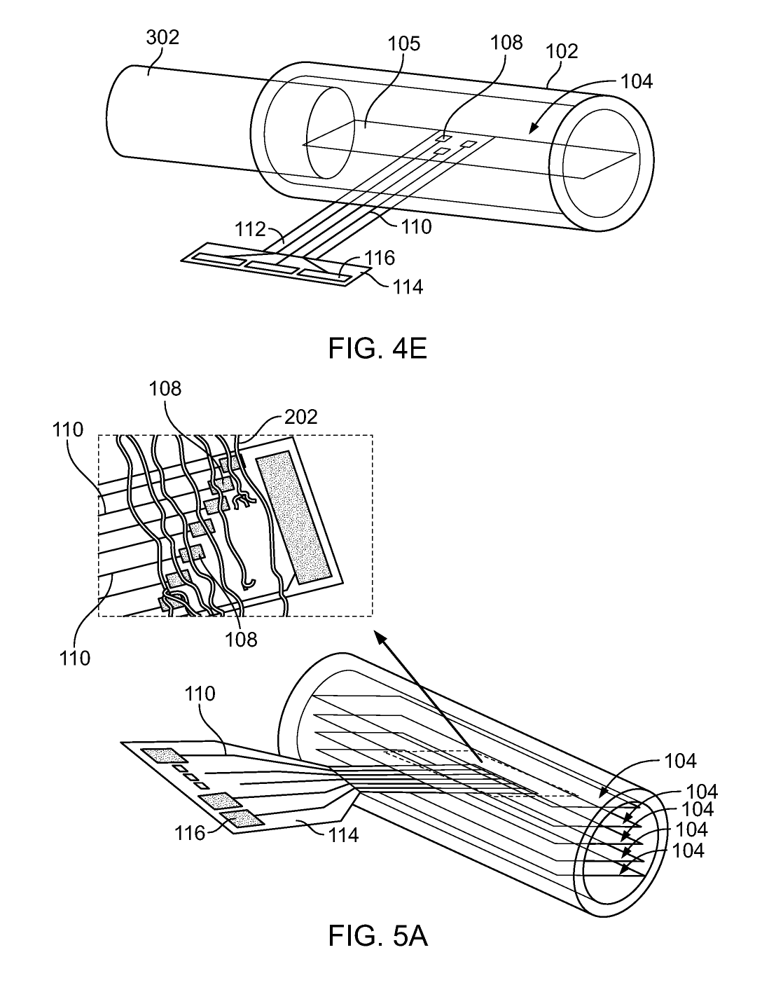

FIG. 4A shows an example of the implantable device positioned between a proximal nerve stump 302 and a target for the regenerating or growing neural tissue 602. In this example, the original target 604 was removed when the nerve was transected. An alternative target 602 can be positioned in the target zone of the implantable device such that neural processes regenerating or growing along the substrate surface can innervate the new target 602. This surrogate or artificial target 602 replaces the original target of the axons. FIG. 4B shows a similar configuration; however, there is a distal portion of neural tissue 702 that innervates the new target 602. Thus, axons growing from the proximal nerve stump 302 along the surface of the substrate contacts and grows into the distal neural tissue 702 and to the new target 602. FIG. 4C shows that neural tissue 704 can be used as a distal target to support axon regeneration. FIG. 4D shows another example where the nerve and axons are directed along the surface of the substrate back to the original distal nerve stump 304 and back to its original target 702. FIG. 4E shows an example with no target tissue to which the axons grow from the proximal nerve stump 302 along the substrate.

FIGS. 5A and 5C show an example configuration where multiple layers of substrate are stacked within a guiding tube or support structure. Each substrate layer can be integrated with an interfacing unit. A stacked structure allows neural tissue to grow along one or more of the stacked layers and make operative contact with electrodes or interfacing units integrated with one or more of the layers. This configuration can be used to increase the surface area for operative communication between interfacing units and neural tissue and can consequently provide higher resolution.

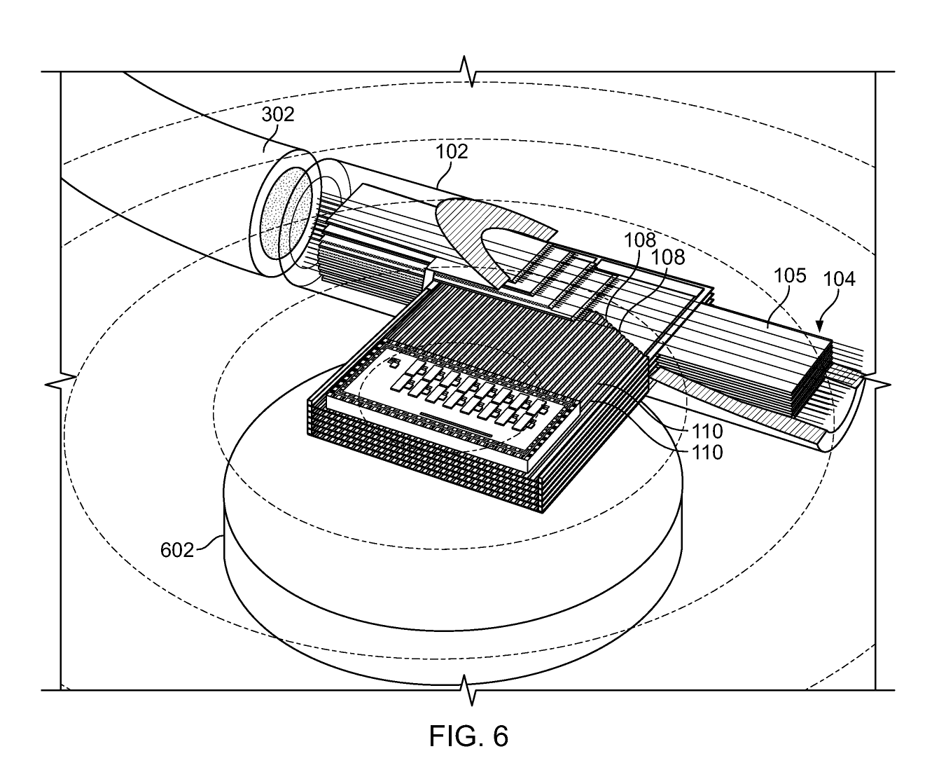

FIG. 6 shows an example implantable device where multiple substrate 104 layers and multiple corresponding interfacing units, for example, electrodes 108, are integrated with the plurality of substrate layers. The layers are supported within a tubular substrate 102 to provide a high order of interfacing sites for high resolution communication with tissue. This configuration still functions to cause low blocking of cross sectional area through a growth area of the nerve stump 302. FIG. 6 further shows a wireless transmitter 602 for transmitting information gathered from the interfacing units for further processing.

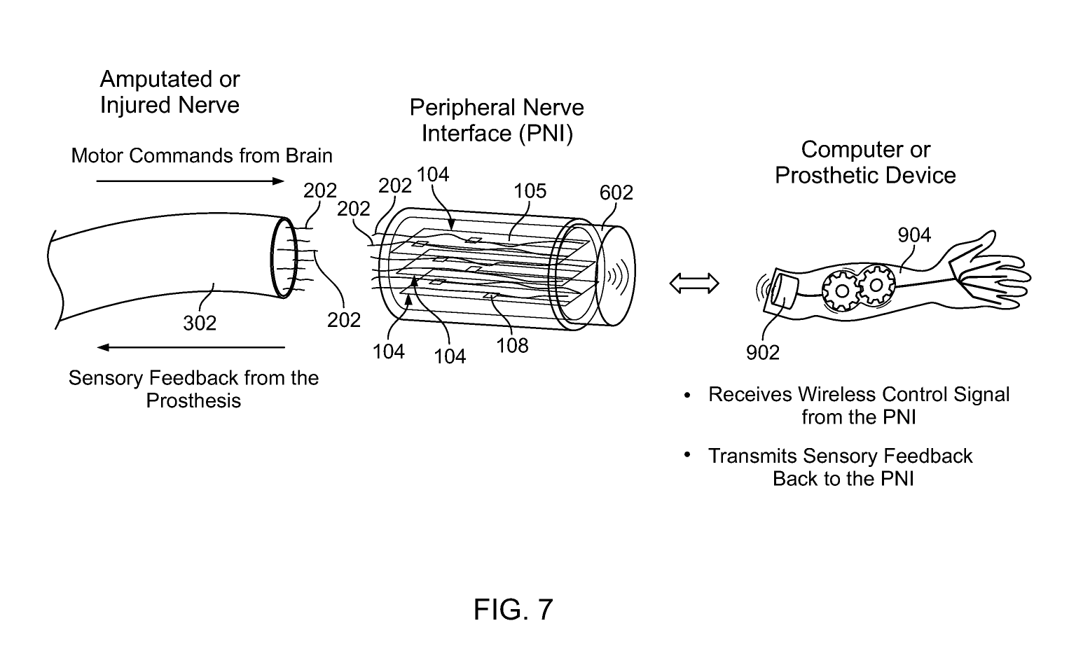

FIG. 7 shows a schematic view of a implantable device with multiple substrate layers 104 and a wireless transmitter 602. The wireless transmitter is in communication with a prosthetic device 904. The prosthetic device can comprise a wireless receiver 902, a processor unit to receive and process signals from the wireless transmitter 602. The received information can be used for controlling one or more actuators disposed within the prosthetic device to control movement of the device.

A proximal nerve stump is also in communication with the implantable device wherein the regenerating or growing axons grow along the substrate surface using topographical cues and make operative communication connections with interfacing units positioned on or relative to the substrate surface. Thus, the prosthetic device can be integrated with the implantable device such that motor commands from the brain can be translated into motion of the prosthetic device. Moreover, sensory information from the prosthetic device can be transmitted to the subject using the implantable device. In this case, if the prosthetic device senses stimulation, the stimulation can be transferred through the implantable device and to a sensory axon, subset of axons, or all axons in a subset or neural tissue that has made an operative communication connection with an interfacing unit that will provide stimulation to a sensory axon or axonal bundle.

In the case of providing motor control to the prosthetic device, a motor command from the brain moving down the proximal stump can be sensed by an electrode in operative communication with an axon or axon bundle of the proximal stump. This electrical activity can be sensed and processed for pattern recognition. Recognized patterns can be interpreted as the desire to move the prosthetic device or portions thereof. Signals to move the desired portion of the prosthetic device based on the pattern can be transmitted from a wireless transmitter to the receiver in the prosthetic device to cause motion of a desired portion of the prosthetic device.

Electrodes or combinations of electrodes can be identified in patterns for performing different actions with a prosthetic limb. For example, certain patterns of electrical activity sensed by the electrodes of the implantable device can be modeled and interpreted as motor commands for moving desired portions or selected portions of a prosthetic device. Similarly, patterns of sensory stimulation in the prosthetic device can be recognized and transmitted to the proximal stump in a particular pattern. These patterns of activity on the electrodes can be interpreted as an appropriate sensory stimulus on the prosthetic device or as an appropriate motor stimulation to manipulate a appropriate portion of the prosthetic device.

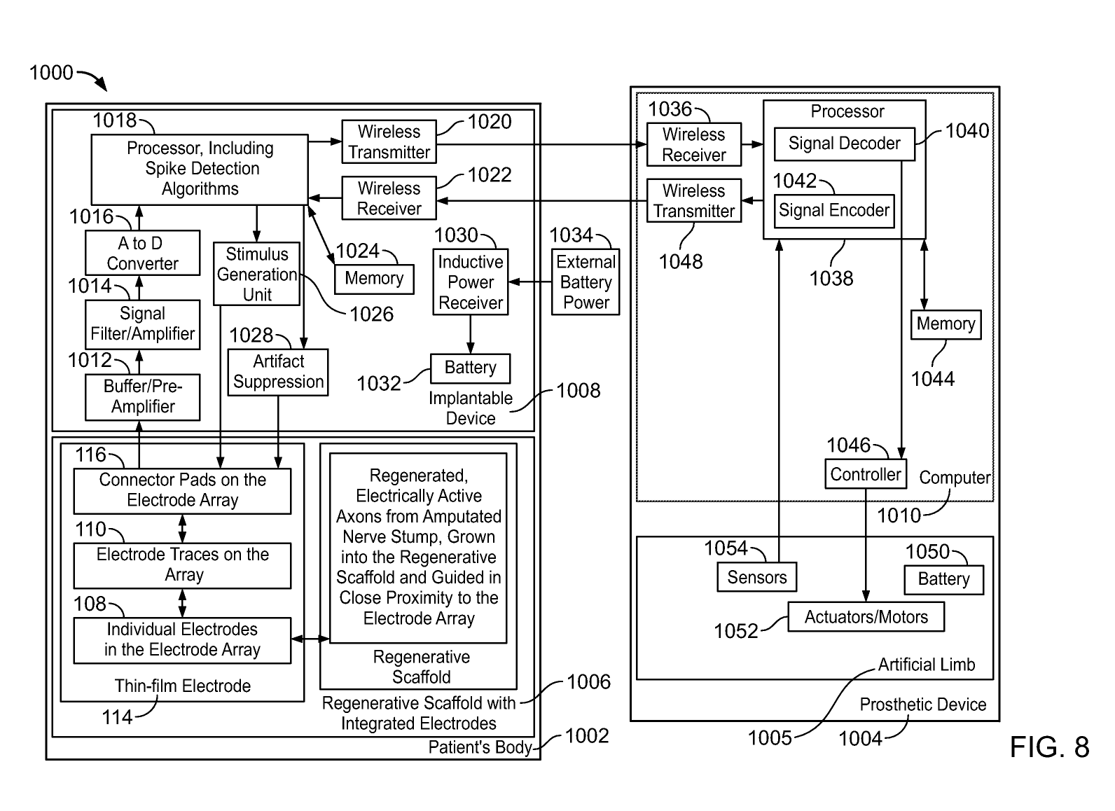

FIG. 8 shows an example system 1000 for use with an implantable device. In this example system, the implantable device 1006 can be implanted into a subject's body 1002 such that regenerating tissue can grow along the substrate surface using topographic cues. The tissue growing along the substrate surface can be directed into operative communication with interfacing units positioned on the substrate 104 or on the surface 105 of the substrate 104. For example, a plurality (i.e., array) of electrodes 108 can be used which can be used to stimulate tissue. The individual electrodes in the array can be a part of a thin film electrode 114. Each individual electrode 108 in the array has an associated trace 110 that is connected to connector pads 116 of the electrode array for communication with other portions of the system.

When the connector pads 116 are connected with other portions of the system, electrical stimulation can be provided through the electrode traces 110 to the individual electrodes 108 and to the tissue to be stimulated. Electrical activity can also be sensed by the individual electrodes in the array 108 and can be transmitted along the electrode traces 110 to the connector pads 116, and to the remainder of the system. For example, the connector pads can be connected with an implantable processing unit 1008 that can comprise a buffer preamplifier 1012 for buffering and amplifying signals from the control pads. Buffered and amplified signal can be further filtered and amplified at block 1014. Analog signals can be converted to digital signals in block 1016. The converted digital signals can be processed by the processor 1018.

The processor 1018 can include spike detection algorithms. The buffer preamplifier 1012 and 1014 can be used to amplify signals, because signals from the individual axon, subset of axons, or the entire set of axons of the neural tissue can be small. Various portions or all of the functions of the implantable processing unit 1008 can be optionally performed outside of the subject using an external device.

Each electrode can be sampled at various frequencies. For example, each electrode can be sampled many times a second. Optionally, sampling can be performed at 5 kHz or more. The spike detection algorithms allow for a subset of the data provided by the analog to digital converter 1016 to be presented to the wireless transmitter for transmitting for further processing. By using the software, the processor unit can transmit when there is a spike detected, thereby reducing the amount of information for wireless transmission. In other example systems, particularly when wireless transmission is not used, spike detection algorithms are optionally not used because a larger bandwidth is possible.

Information directed to the wireless transmitter 1020 can be transmitted to wireless receiver 1036 for further processing by a second processor unit 1038. Processor unit 1038 can decode the pattern of spiking and determine through classification algorithms what the subject's original intention was, based on the mapping performed and stored in memory 1044. The processor uses algorithms to compare the received spikes with previously mapped pattern definitions to determine what the received spike patterns represent. Having identified a spike pattern with the previous mapped patterns, the prosthetic device 1004 can be controlled using the controller module 1046 that is in communication with actuators or motors 1052 of the prosthetic device.

The prosthetic device can also comprise sensors 1054 which are in communication with a signal encoder 1042. Sensory patterns detected by the sensors 1054 can be used to determine the sensory stimulus location type or intensity at the sensor of the prosthetic device. For example, proprioceptive feedback after movement of the prosthetic device can be detected. Information regarding sensory patterns can be transmitted from the wireless transmitter 1048 to the wireless receiver 1022.

The processor unit 1018 can be used to generate the stimuli necessary to create the feedback to the subject. The stimulus generation unit 1026 is in communication with the control pads, which in turn are in communication with the electrode array can stimulate the sensory pathway alerting the individual about sensory information detected at 1054. Based on the mapped patterns, stimulus generation stimulates the correct pattern of sensory axons, subsets of axons, or all axons of neural tissue in operative communication with the array electrodes to simulate that sensory feeling such as propreioceptive, pressure or other sensory information. On the feedback loop, the processor 1018 decodes information on the stimulus patterns into the appropriate signals for stimulating the electrodes.

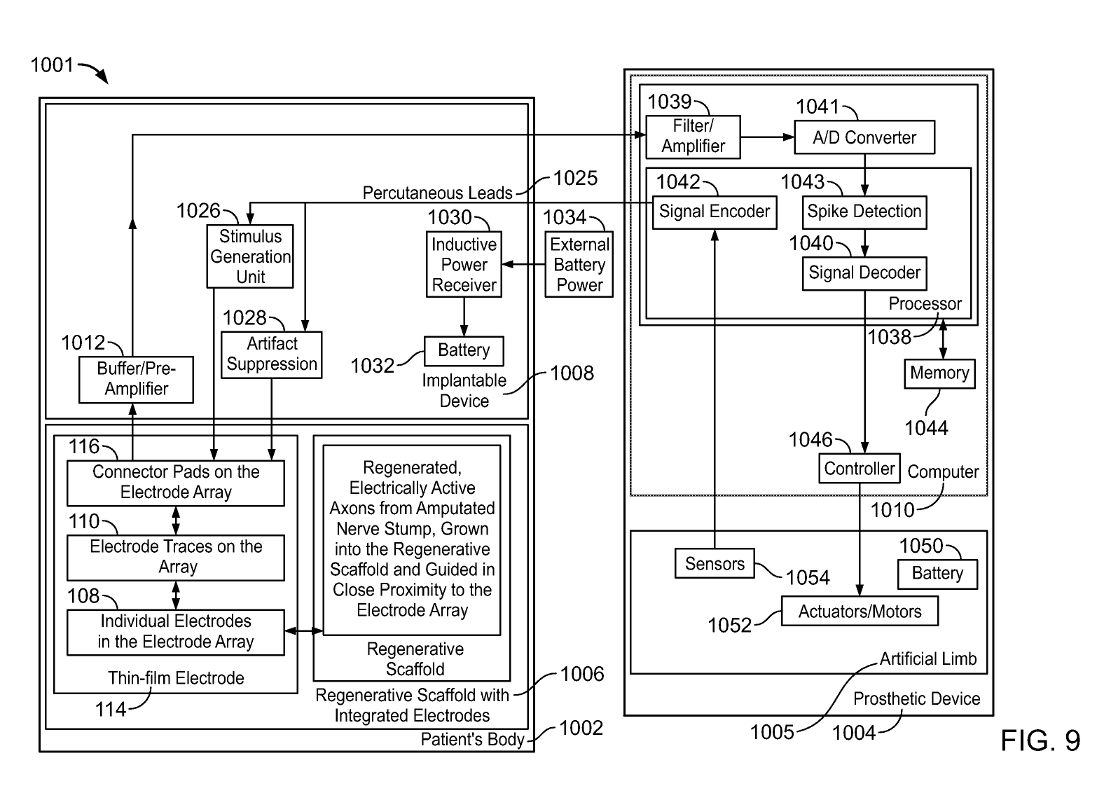

FIG. 9 shows an example system 1001 where wireless transmission is not used. Instead of wireless transmission, percutaneous leads 1025 are used to communicate between the implantable device and the processing unit 1038. In each example system described, common power sources can be used. For example, an inductive power receiver 1030 can be located within implantable device 1008 that can charge a battery 1032 for powering the processor and other portions of the implantable device from an external energy source 1034. Similarly, the prosthetic device can comprise a battery unit 1050 for powering the actuator motor and sensory or other components of the prosthetic device 1005.

Examples of implantable devices include and are referred to herein as scaffolds. The term scaffold includes regenerative nanoscaffolds, nanofiber scaffolds and regenerative electrode scaffolds. Thus, when these terms are used they refer to non-limiting examples of implantable devices as described throughout. These examples of devices can comprise a substrate positioned in relation to a support structure. These examples of devices can further comprise an interfacing unit positioned relative to the substrate for communication with tissue grown or regenerated along the surface of the substrate.

### EXAMPLES

The following specific examples further illustrate the invention.

### Example 1

Oriented nanofiber films, which can be used to provide a substrate as described herein, support robust, oriented neurite extension. Contact guidance based substrates for peripheral nerve repair were developed. Polymeric (polysulfone) nerve guidance channels were used featuring an interior substrate of layered films of oriented polymeric nanoscale fibers. Layers of oriented nanofibers (PAN-MA (poly acrylonitrile-co-methylacrylate); 200-800 nm diameter) were created through an electrospinning process, as depicted in FIG. 1B.

The PAN-MA solutions were prepared by dissolving polymer pellets into DMF (dimethylformamide). The solution was then loaded into a syringe with a feeding rate precisely controlled by a syringe pump. A high speed rotating metal drum was placed near the syringe tip, and a voltage of 22 kV was applied between the syringe needle and the metal collecting drum. As the PAN-MA was slowly ejected from the needle tip, the strong electric field helped to generate fine polymer fiber jets that stuck to the metal drum in an aligned fashion due to its high speed rotation. The fiber jets were collected for a set length of time to create 10 μm thick film layers of oriented 200-800 nm PAN-MA fibers.

To assess the ability of an oriented nanofiber layer substrate to direct neurite outgrowth in vitro, whole dorsal root ganglia (DRG) were cultured on top of a film of oriented nanofibers. The majority of neurite outgrowth from the DRG neurons extended parallel to the oriented nanofibers. Schwann cell migration and laminin deposition preceded the extending processes in vitro, and neurites were found to be co-localized with migrated Schwann cells. These results demonstrated that that oriented nanofibers guided the extension of DRG neurites in part by first facilitating Schwann cell migration along the fibers. This strategy of guiding axonal growth with topographical cues is used to direct regenerating axons across integrated electrodes of the substrate.

Nanofiber based substrates supported robust in vivo regeneration of severed peripheral nerves. Oriented nanofiber films were cut into rectangular strips and stacked in layer inside a semi-permeable polysulfone tube. A sciatic nerve gap of 17 mm was used to create an in vivo model of peripheral nerve defects. When a 17 mm gap was bridged with a saline filled polysulfone tube, no regeneration occurred. A length of the tibial nerve was resected, and the proximal and distal stumps of the nerve were sutured into either end of a 19 mm polysulfone guidance channel filled with 15 layers of nanofiber films (the resulting gap is 17 mm, because the nerve stumps are sutured 1 mm into the ends of the tube).

The implanted device facilitated the regeneration of transected tibial nerves across the gaps. The transected axons entered into the proximal end of the tube, regenerated through its entire length along the nanofiber substrates, and moved into the distal stump of the nerve. The regenerated axons and the infiltrated Schwann cells grew along the aligned nanofiber substrates, showing that the oriented films guide the direction of the regenerating axons and infiltrating Schwann cells after injury, as shown by the in vitro observations of DRG neurite extension.

In addition, cross-sectional images reveal that the regenerating axons co-localize with the infiltrated Schwann cells, which deposit myelin through the length of the scaffold. No axons were observed in the absence of Schwann cells. These nanofiber based implantable devices performed as well as autografts in a variety of tests, including histological, electrophysiological, behavioral assessments. Thus the aligned fibers can facilitate nerve regeneration across long nerve gaps even in the absence of exogenous proteins or trophic factors by facilitating Schwann cell migration.

An example implantable device was designed with a linear array of active sites spaced 100 μm apart in one dimension and 50 μm apart in the other dimension. The device thickness was 85 μm, which included a 50 μm polyimide substrate, a 9 μm conductor layer, a 1 μm gold active layer, and a 25 μm polyimide cover layer. The array was characterized in saline using an impedance spectroscopy system.

Another example implantable device was fabricated using integrated circuit (IC) fabrication techniques. IC CAD tools were used to lay out the thin-films with feature sizes on the order of 10 μm, and a set of devices was fabricated on four inch diameter silicon wafers. These thin-film electrodes have an overall thickness of 16.35 μm including a 12 μm polyimide substrate, a 2 μm titanium/gold/titanium conductor layer, a 0.35 μm iridium oxide active area, and a 2 μm polyimide cover-layer. Iridium oxide was sputtered onto the film. A commercially available device from Multichannel Systems (Reutlingen, Del.) can also be used. This commercial device contains 30 μm diameter titanium nitride active areas spaced 300 μm apart in a grid array on a 12 μm thick film.

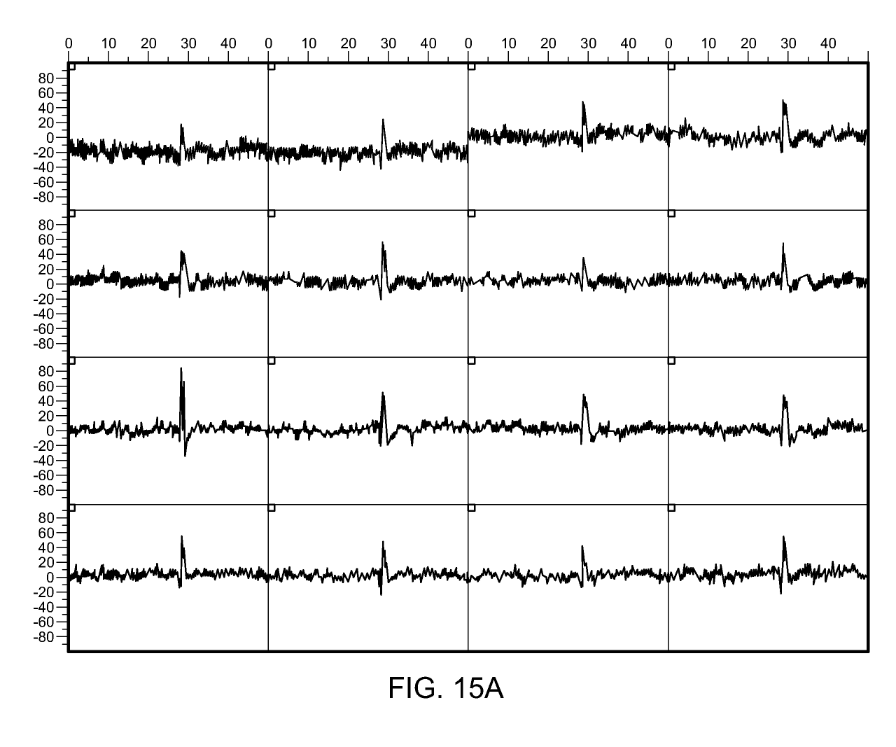

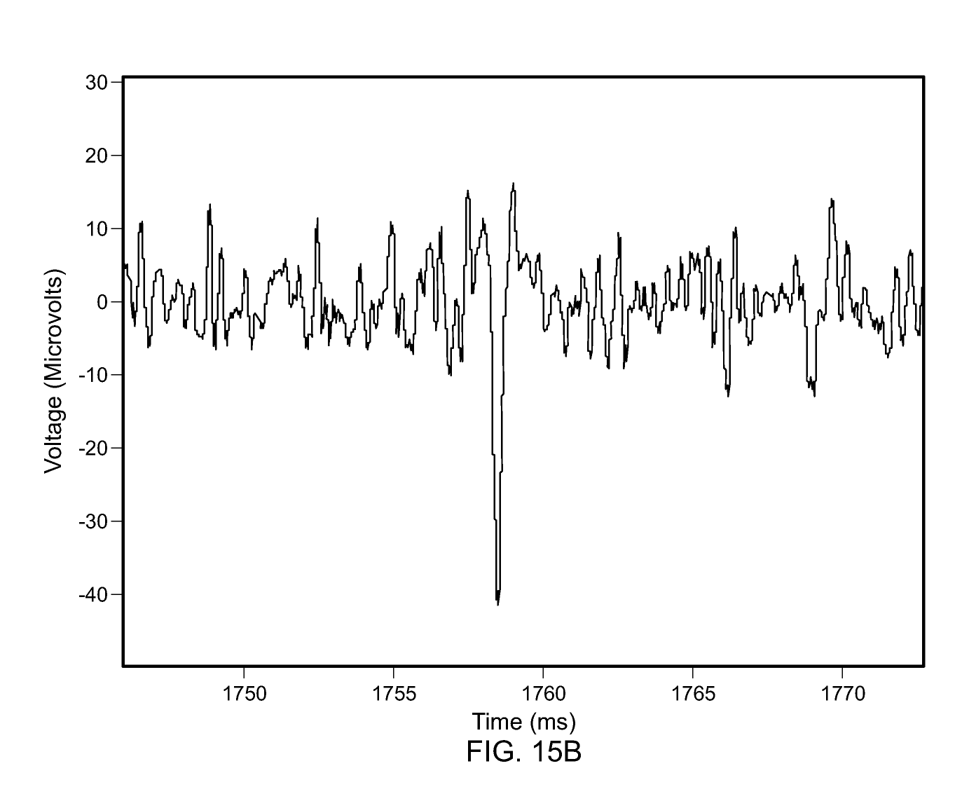

Thin-film electrodes recorded signals from primary neurons in vitro. Integrating thin-film electrodes into the nanofiber film substrate enabled high probability co-localization of nanofiber guided regenerating axons and electrode sites. A commercial 32 channel thin-film electrode was used in vitro, and recordings were taken using an invertebrate model. A severed connection from the abdominal ganglion of an Aplysia slug was draped across the active end of the electrode, and spontaneous spiking was recorded (FIGS. 15A and 15B). Extracellular action potentials were recorded on all electrode sites. Additionally, the thin-film electrode was used on a rat tibial nerve in which a segment of the epineurium was removed. Bursts of action potentials on all channels were detectable, as well as individual spikes.

A short distal nerve segment was used for robust neurite extension through the implantable device with integrated thin-film electrodes. A short 2-3 mm nerve segment derived from the same animal was sutured in as a substitute distal stump to provide a distal source of Schwann cells. The nerve segment was terminal and not connected to any tissue/end organ. Additionally, to evaluate potential growth across an integrated electrode array, a polyimide electrode (titanium nitride) was integrated within in the substrate.

In each procedure, rat sciatic nerves were sectioned and all distal connections were resected up to their attachment points. Four weeks after implantation, the substrates were explanted, longitudinally cryosectioned, and stained for axons, and Schwann cell markers. The results demonstrate that even in the absence of the original distal target, the substrate is able to support robust regeneration through the entire length of the substrate and across an electrode array site. At the time of explantation, the regenerated distal end of the nerve segment remained isolated and unconnected to any of the surrounding tissue.

An abundance of axons and Schwann cells were seen growing in close proximity to the surface of the array. These results demonstrate the ability of the substrate to foster healthy initial regeneration in the absence of the distal stump, with just a small nerve segment presumably providing a distal source of Schwann cells.

### Example 2

### A. In-Vitro

Regenerative nanoscaffolds, an example of an implantable device, comprising at their core of layers of oriented nanofibers (PAN-MA, poly acrylonitrile-co-methylacrylate, 200-800 nm diameter) were prepared. These oriented nanofiber films, 10-μm thick, were produced using an electrospinning process. A high voltage (20 kV) was applied between a syringe as it slowly ejected a liquid polymer melt and a high speed rotating metal drum. Fibers ejected from the syringe were collected on the rotating drum and extracted in oriented sheets.

Whole dorsal root ganglia (DRGs) from postnatal day 3 (P3) rat pups were extracted and seeded on top of a sheet of oriented nanofiber film that was secured at the corners to the bottom of a Petri dish with biocompatible glue. Growth was assessed not only on the nanofiber layer but also across a nanofiber/polyimide boundary onto a 12 μm thick polyimide sheet, chosen to simulate the surface of a polyimide electrode array.

The nanofiber/polyimide interface was created in several different configurations. These design conditions included whether the nanofiber layer was overlapped, underlapped, or laid flush to the polyimide boundary. Other conditions included the presence or absence of surface modification to the polyimide surface, such as directional scratches or addition of extracellular matrix (polylysine or laminin).

DRGs were seeded on the nanofiber sheet 1-5 mm from the polyimide boundary and after 10-14 days the dishes were fixed and immunostained for axons (nanofilament 160 kD NF-160), Schwann cells (S-100 protein), and cell nuclei (4′,6-diamidiro-2-phenylindole DAPI). Migration of Schwann cells and extension of neurites through the nanofibers and across the polyimide boundary was assessed using a fluorescent microscope.

### B. Regenerative Electrode Scaffold (RES) Fabrication

The scaffolds were fabricated by stacking layers of PAN-MA nanofibers within a polymeric (polysulfone) tube. The integration of thin-film electrode(s) into individual layers of nanofiber scaffold was used to create a RES, a device capable of establishing a stable, high resolution, peripheral nerve interface.

A nanofiber scaffold containing a single nanofiber or electrospun layer affixed down the mid-horizontal plane of the tube, with a polyimide electrode array embedded within the layer at the center of the tube was used. Results of the in-vitro DRG culturing experiments were used to optimize the techniques for integrating the polyimide electrode (2 mm×2 mm active area) into the center of the scaffold. In most cases, a non-functional electrode, consisting of the polyimide substrate alone, was used to make the RES's. After fabrication, tubes were UV sterilized overnight and stored in sterile saline until implantation.

### C. In-Vivo

RES's were fabricated to accommodate nerve gaps of 6, 10, and 13 mm. Initial surgeries were performed on 6 anesthetized Fischer 344 rats (250-300 g), 2 rats per gap length. The sciatic nerve was exposed, and the tibial nerve was transected several millimeters distal to the tibial/common peroneal bifurcation. The proximal and distal stumps of the cut nerve were then secured into either end of the RES with 10-0 sutures.

The nerve was allowed to regenerate through the scaffolds for periods of 3-6 weeks, (although more time was allowed for regeneration to occur through the longer scaffolds), and the rats were perfused transcardially with a 4% paraformaldehyde mixture. The scaffolds were then explanted and prepared for cryosectioning in a 30% sucrose solution. 18 μm thick longitudinal sections were obtained with a cryostat, collected on glass slides, and double immunostained with markers for axonal regeneration and Schwann cell migration (NF-160 and S-100 staining). On some samples, double staining was performed using antibodies S-100 and either ED-1 or vimentin, for macrophages and fibroblasts/macrophages, respectively.

### D. Further In-Vivo Activity

Two additional blind-ended implantations, in which no intact distal nerve stump was present, were performed to better simulate the amputation case. In these blind-ended cases, all procedures were the same, except that after nerve suturing, the distal portion of the tibial nerve was cut and resected up to the muscles. Only an isolated fragment of nerve (2-3 mm) was left at the end of the tube to supply a source of migrating Schwann cells. The nerve gap in these cases was chosen to be 6 mm, based on the results of the first experiments.

Also, two rats were implanted with RES's containing functional 32-channel microfabricated polyimide electrode arrays containing gold traces and 30 μm² electrodes coated with titanium nitride (Multichannel Systems, Reutlingen, Del.) The gap length in these implants was 6 mm.

Results

A. In-Vitro DRG Cultures

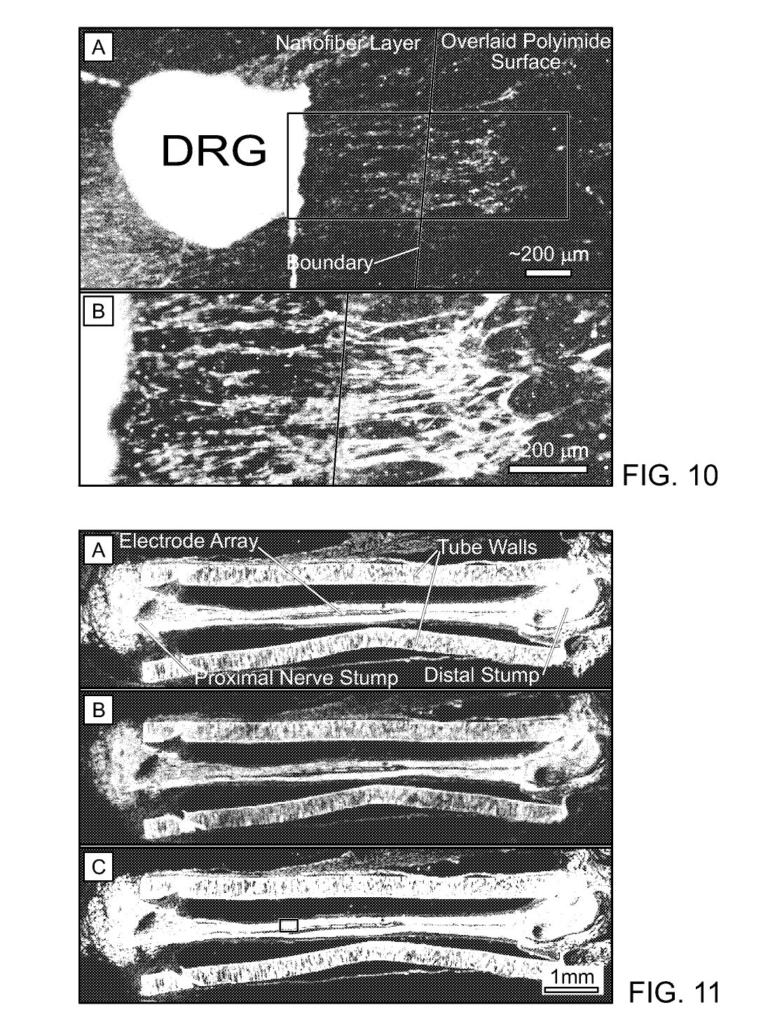

DRGs cultured on a nanofiber layer adjacent to a polyimide surface demonstrated the ability to extend neurites and migrating Schwann cells along the nanofibers and across the nanofiber-to-polyimide boundary. FIG. 10 shows an example of robust Schwann cell migration from a DRG through the oriented nanofiber layer and across an overlaid polyimide surface. Oriented neurite extension through the nanofibers was strong as well, but extension of neurites onto the polyimide surface was relatively weak as compared to the observed Schwann cell migration across the same boundary. However, many examples of boundary crossings by extending neurites were observed, demonstrating in vivo axonal regeneration along oriented nanofibers and onto an integrated polyimide electrode array site.

In vitro culturing data showed that the right proportion of biocompatible adhesive can be used to enhance growth from the nanofiber layer up and onto an overlaid polyimide surface.

### B. In-Vivo Implants

In all, 10 rats implanted with a RES, localized Schwann cell migration and axonal regeneration through the length of the scaffold and across the embedded polyimide layer was observed. In the 6 initial implants, in which the nerve gap lengths were varied, this regeneration was robust, even for the longest nerve gap length of 13 mm. Additionally, in all cases, regeneration was localized almost exclusively in regeneration cables on either side of the nanofiber layer. This localization occurred even for the shortest nerve gap lengths of 6 mm. This finding demonstrated the strong preference of regenerating nerves for the nanofiber layer surface. A gap length of 6 mm was used in subsequent experiments for simplicity, but localized, directed growth through even a shorter gap occurs.

FIG. 11 shows different fluorescent staining of a longitudinal section obtained from a typical RES. This particular RES was explanted after 4 weeks and contained a functional polyimide electrode array. FIG. 11A shows Schwann cell migration through the scaffold, and FIG. 11B shows axonal regeneration. These images demonstrated that the configuration of the RES enabled directed axonal regeneration in a localized fashion through the mid-horizontal plane of the scaffold, along the substrate and across the embedded electrode. FIG. 11C, which shows a merged image of FIG. 11A and FIG. 11B and demonstrated the co-localization of axons and migrated Schwann cells through the length of the RES.

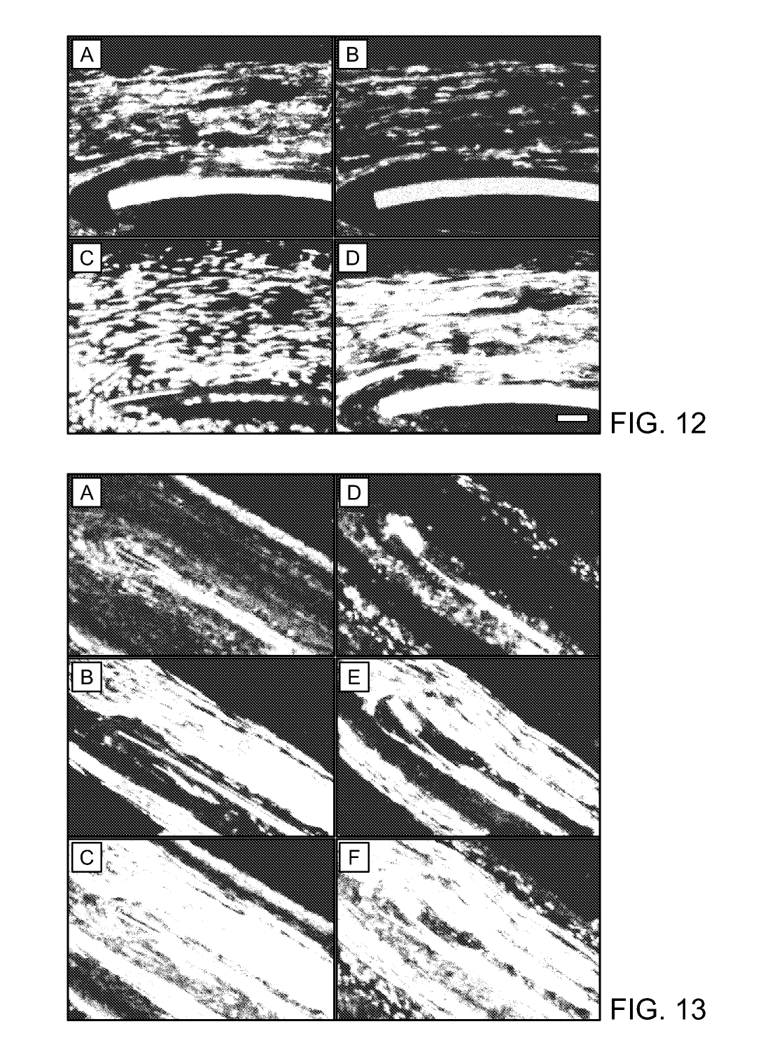

FIG. 12 provides higher magnification of the proximal end of the of the electrode array (the small white boxed region in the center of FIG. 11C) and demonstrated the close proximity of directed axonal regeneration to the array surface. DAPI staining, shown in FIG. 12C, indicated that the space that exists between regenerated axons and the electrode surface is cellular in nature. FIG. 13 confirmed this finding and characterized this tissue as consisting of macrophages and fibroblasts. This observed inflammatory response was minimal and was characteristic of any implanted foreign object within the body. The semipermeable nature of the polysulfone tubes used in our RES's (molecular weight cutoff of 50 kD) apparently limit the inflammatory response during nerve regeneration. Within all implanted RES's, separation distances between regenerated axons and the electrode surface were small, varying from as low as several microns to a few tens of microns at most.

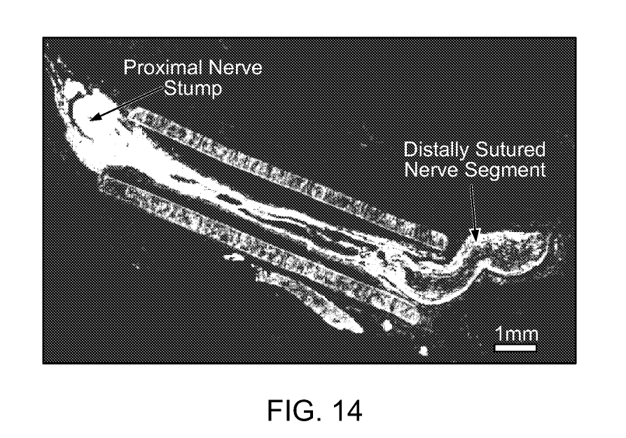

In the rats implanted with scaffolds attached to only a nerve fragment at the distal end (as opposed to the intact distal stump), healthy regeneration was again observed. In the other animal, regeneration was found to be as robust as in the best intact distal stump cases (see FIG. 14).

### Example 3

Aligned fiber films of poly(acrylontrile-co-methylacrylate, random copolymer, 4 mole percent of methylacrylate) (PAN-MA) were created through an electrospinning process. A 15% (w/v) PAN-MA solution was prepared by dissolving PAN-MA into the organic solvent, N,N-Dimethyl Formamide (DMF, Acros Organics, Geel, Belgium) at 60° C. This solution was loaded into a metered syringe and dispensed for 15 minutes at a constant flow-rate of 1 ml/hr through a 19 gauge needle across a high voltage field (15-18 kV). The ejected polymer fibers were collected 10 cm away on a high speed rotating metal drum to form aligned films, which were later baked for 4 hours at 60° C. to remove any residual DMF. Finally, 2.2×14 mm sheets of aligned fiber films were cut manually with a razor and peeled off with fine forceps for use in scaffold construction. Film samples were also collected for characterization with bright field and scanning electron microscopy (LVEM5, Delong Instruments, Brno, Czech Republic).

Polysulfone nerve guidance channels (Koch Membrane Systems, Wilmington, Mass.) were used to contain the oriented fiber film scaffolding. The semipermeable polysulfone tubing (inner diameter: 1.6 mm; outer diameter; 2.2 mm, molecular weight cutoff: 50 kDa) was first cut into tubes of 17 mm length. These tubes were next sectioned lengthwise into 4 longitudinal sections, using a machined aluminum template.

A manual layer-by-layer approach was then used to fabricate the scaffolds, with each layer secured into place with a medical grade UV light curing adhesive (1187-M-SV01, Dymax, Torrington, Conn.). In the 1-film case, a single thin-film was secured longitudinally through the length of the tube. In the 3-film case, two additional films were fixed through the tube, but distributed from each other in a ‘Z’ formation. Notably, the 1-film guidance channels could have been constructed more simply by splitting the polysulfone tubes longitudinally into two pieces rather than four, but to fabricate all scaffolds were fabricated in the same fashion to minimize variability.

The scaffolds were sterilized by overnight incubation under a UV light and then immersion in 70% ethanol for 30 minutes. This process was followed by two 20 minute washes in sterilized deionized water, and a final wash in sterilized phosphate buffered saline (PBS). The scaffolds were then stored in PBS until the implantation surgery.

Implantations to bridge 14 mm gaps in sciatic nerve were performed on 30 anesthetized Fischer 344 rats (250-300 g). The rats were anesthetized with inhaled isoflurane gas, and the surgical site was shaved and sterilized. Marcaine (0.25% w/v, Hospira, Inc., Lake Forest, Ill.) was next administered subcutaneously for post-surgical pain relief (0.3 ml/rat). A skin incision was then made along the femoral axis, and the underlying thigh muscles were delineated with a blunt probe to expose the sciatic nerve. After the nerves were freed from overlying connective tissue, microscissors were used to transect the tibial nerve branch, slightly distal to the common peroneal-tibial bifurcation, and the nerve stumps were pulled 1.5 mm into each end of the guidance scaffold and fixed into place with a single 10-0 nylon suture (Ethilon™, Ethicon Inc., Piscataway, N.J.).

The muscles were then reapposed with 4-0 vicryl sutures (Ethicon Inc., Piscataway, N.J.) and the skin incision was clamped shut with wound clips (Braintree Scientific, Inc., Braintree, Mass.). After the surgery, the rats were placed under a warm light until stable, and then housed separately with access to food and water ad libitum in a colony room maintained at constant temperature (19-22° C.) and humidity (40-50%) on a 12:12 h light/dark cycle. To prevent toe chewing, a bitter solution (Grannick's Bitter Apple™, Valore Chemical Corp., Greenwich, Conn.) was applied twice a day to the affected foot. When further action was required, treatment with a mixture of New Skin™ (Prestige Brands, Irvington, N.Y.) and Metrozodinial™ (ICN Biomedical Research Products, Costa Mesa, Calif.) proved highly effective.

At time points of 6 weeks (10 animals) and 13 weeks (20 animals), rats were evaluated for nerve regeneration. Each time point consisted of two groups, one receiving 1-film scaffold implants and one receiving 3-film scaffold implants. The 6 week time point was chosen to provide an early view of the regenerative process, but only after allowing an appreciable degree of axonal regeneration to occur through the full length of the guidance scaffolds. Regeneration in the 6 week group was quantified with histological measures alone.

The 13 week time point was chosen to allow for an appreciable degree of functional recovery to take place. Accordingly, additional evaluation measures were taken at this time point, including nerve conduction velocity, muscle force production, relative gastrocnemius muscle weight (RGMW), and staining of neuromuscular junctions. It is significant to note that the process of electrophysiological testing, perfusion, and tissue harvest took several hours per animal, and so this phase of evaluation spanned a period of approximately 10 days as scheduled. As a result, the exact regeneration times were actually 12.5-14 weeks, but for simplicity this time point is elsewhere referred to as the 13 week time point. To prevent bias in total regeneration times between groups, evaluations were performed with animals from different scaffold type groups tested in alternating fashion.

In order to assess functional recovery, a set of electrophysiological measures were conducted on each rat, including conduction velocity (CV) of compound action potentials (CAPs), maximal muscle force production, and EMG response of the muscles. Each animal was deeply anesthetized with a mixture of ketamine (65 mg/kg), xylazine (7.5 mg/kg), and acepromazine (0.5 mg/kg), and a catheter was sutured into the intraperitoneal (IP) space to allow continued dosage during the evaluation. The site of nerve injury was exposed as during the initial surgery, and the cavity was kept moistened with mineral oil warmed to 37° C. Through the procedure the animals were kept warm with an infrared light, and their breathing rates and reflex responses to toe pinches were closely monitored.

A portion of the sciatic nerve, approximately 15 mm proximal to the beginning of the scaffold, was freed from the surrounding tissue, as was a portion of the distal tibial nerve branch, approximately 15 mm past the distal end of the scaffold. Stainless steel bipolar hook electrodes were fixed to both exposed portions of nerve, approximately 45 mm apart. The distally positioned electrodes, attached to a stimulator (Model S88, Grass Technologies, West Warwick, R.I.) and SIU (Model SIU5B, Grass, West Warwick, R.I.), were used to stimulate the regenerated nerve with triggered 100 μs square pulses of variable amplitude, applied at a rate of 1 Hz. The evoked CAPs were recorded by the proximal electrodes, where they were amplified (G=1000), bandpass filtered (10-5000 Hz, Model 1700, A-M Systems, Sequim, Wash.), and digitally sampled using a 25 kS/sec, Multichannel Systems DAQ card (Reutlingen, Del.). Recordings were averaged up to 200 times and the latency of the onset of the evoked CAP was determined off-line. The precise separation distances between stimulating and recording electrodes was carefully measured (approximately 45 mm in most cases), and used to calculate the conduction velocity of the CAPs through the regenerated nerves.

As another functional test of regeneration, muscle force measurements were performed. In each case, the lateral and medial gastrocnemius muscles were exposed and tied off with a silk thread at the distal tendon, which was then cut from its insertion point. The thread was tied at the other end to a force transducer (LCL-227G, Omegadyne Inc., Sunbury, Ohio), which was in turn connected to an amplifier (Model 440, Brownlee Precision Co., San Jose, Calif.) attached to the same DAQ card. The gastrocnemius muscles were separated from the surrounding tissue, and the knee was firmly immobilized with a clamp. 100 μl sec stimulus trains, composed of supramaximal square pulses repeated at 150-200 Hz, were applied to the regenerated nerve, and the resulting force deflections produced by the gastrocnemius muscles were recorded and stored for off-line analysis. This testing was repeated across a range of muscle lengths to ensure that the muscles were at their optimal lengths. As part of this testing, a curve relating the baseline passive muscle forces versus corresponding active tetanic muscle force was generated for each rat. To ensure nerve signals were passing through the regenerated fibers, nerves were crushed immediately distal to the stimulation site, and it was verified that no muscle twitches resulted.

After electrophysiological evaluation, the rats were perfused intracardially with saline followed by 4% parafomaldehyde (Sigma-Aldrich, St. Louis, Mo.) in PBS. The injury site was fully exposed, and the nerve guidance scaffolds were explanted for histological analysis. The gastrocnemius muscles from the experimental and control side were also explanted, and all harvested tissues were post-fixed overnight in 4% paraformaldehyde. The tissues were later washed and stored for several hours in PBS and then transferred to a 30% sucrose in PBS solution for 1-2 days until saturation. Finally, the samples were embedded in O.C.T. gel (Tissue Tek™ (Sakura, Tokyo, JP)) and frozen for cryosectioning (CM30505, Leica, Wetzlar, Del.). 10 μm cross sections were collected from 8 distances through each scaffold. In several scaffolds, 18 μm thick longitudinal cross-sections were instead collected to provide an alternate perspective of regeneration through the scaffolds.

Sections later were immunostained for markers for 1) regenerated axons (anti-NF 160, Sigma-Aldrich, St. Louis, Mo.); 2) Schwann cells, (anti-S-100, Dako, Glostrup, Denmark); 3) myelin (anti-PO, Chemicon Intl., Tremecula, Calif.); 4) macrophages (ED-1, anti-CD-68, Serotec, Oxford, UK); 5) fibroblasts: double stain with anti-vimentin (Sigma-Aldrich, St. Louis, Mo.) and anti-S-100 (to help differentiate non-specific staining of Schwann cells). Sections were all labeled with the following secondary antibodies: Goat anti-rabbit IgG Alexa 488/594, and goat anti-mouse IgG1 Alexa 488/594 (Sigma-Aldrich, St. Louis, Mo.).

Sections were incubated 1 hour at room temperature in a blocking solution of goat serum (Gibco™, Invitrogen, Carlsbad, Calif.) in PBS, incubated overnight at 4° C. in a mixture of primary antibody and blocking solution, then washed and incubated once more for 1 hour at room temperature in a solution of secondary antibody mixed in 0.5% triton in PBS. Finally, slides were washed once more, then dried and coverslipped for evaluation. Some slides were also stained with Masson's Trichrome staining and H&E staining.

Nerve regeneration was evaluated at the center of the gap (7 mm) and (2.5 mm) from each nerve stump by quantifying a) the total number of myelinated axons, b) the area of axonal regeneration, c) the number of myelinated axons per unit area (density), d) the diameter distribution of regenerated axons for each group, e) the thickness of myelination in each group, and f) the area of Schwann cell migration in each group. One-way ANOVA was used for statistical comparison of the various groups, and a p-value<0.05 was considered as statistically significant.

To quantify the number of axons in a given scaffold cross-section, the following technique was used. First, a confocal microscope (LSM 510, Zeiss, Oberkochen, Del.) was used to image a representative subset of the regeneration cable at 40× magnification. The number of NF-160+axons was quantified with Image Pro™ software (Media Cybernetics, Bethesda, Mass.) and used to calculate axonal density for each scaffold. Next, a composite 40× image of the entire regeneration cable cross section was obtained, using a microscope equipped with a computer controlled stage and Neurolucida™ software (MBF Bioscience, Williston, Vt.). Image Pro™ software was then used to precisely quantify the area of axonal regeneration in the entire scaffold, and this area was multiplied by the calculated axonal density to result in a final axonal count. Accuracy of this technique was initially validated by comparing results with manual hand counts, and reproducibility/precision was demonstrated by repeating quantifications on sequential sections.

Because the gastrocnemius muscles are innervated by branches from tibial nerve, they begin to atrophy soon after denervation. To measure the reverse of this atrophy by successful reinnervation, the gastrocnemius muscles from the experimental (right) and control (left) limbs were explanted after perfusion. Tendons were carefully stripped, and the weights of the muscles were measured and used to calculate relative gastrocnemius muscle weight (RGMW). The RGMW, which is defined as the ratio of the muscle from the experimental side to the control side, was used as a measure of motor function recovery. The RGMW should increase following the sciatic nerve regeneration and successful reinnervation of the muscle.

After weighing, gastrocnemius muscles were cryoembedded in a process similar to the scaffolds, and longitudinally cryosectioned into 25 μM thick samples using a cryostat. Tissue sections were collected from the center of the muscle where the cross-sectional area was the highest and triple stained for the following markers: neurofilament 160 (NF160, Sigma-Aldrich, St. Louis, Mo.), synaptic vesicles 2 protein (SV2, Developmental Studies Hybridoma Bank, Iowa City, Iowa), and acetylcholine receptors (using alpha-bungarotoxin-tetramethlyrhodamine, Sigma-Aldrich, St. Louis Mo.). Co-localization of these markers indicates both morphological and functional reinnervation of the neuromuscular synapses. Healthy contralateral gastrocnemius muscles taken from the control limb were evaluated as positive controls, in which a very high degree of stain co-localization would be expected.

Electrospun films of oriented PAN-MA polymer were imaged with a scanning electron microscope. Average diameters of the individual aligned fibers fell into the range of 400-600 nm. The thickness of the oriented films was measured by observing scaffold cross-sections under bright field microscopy. Film thicknesses were seen to be uniform, measuring approximately 7 μm.

All 30 rats survived the scaffold implantation surgery without serious complication and most animals exhibited only minimal autophagia as a result of sensory impairment. At the time of explantation, all guidance scaffolds were found to be structurally intact with the tibial nerve still firmly secured on each end.

Explanted scaffolds were cryosectioned and stained for histological analysis. Immunostained cross-sections revealed substantial axonal regeneration through the full lengths of both scaffold types, at both the 6 wk and 13 wk time points. In all cases, regenerating axons were seen to be co-localized with Schwann cells and located within a regeneration cable comprised additionally of fibroblasts, macrophages, and endothelial cells.

The oriented thin-films remained intact and fixed into place within the scaffold interiors. The thin-films appeared to have influenced the positioning and morphology of the regeneration cables, resulting in characteristic patterns of regeneration within the 1-film and 3-film scaffold types. In general, regeneration cables in the 1-film scaffolds were centered around the single thin-film and contained a single centralized core of co-localized axons and Schwann cells surrounded by collageneous tissue rich in fibroblasts. The 3-film scaffolds featured larger regeneration cables that surrounded all 3 thin-films. These regeneration cables appeared less organized and contained multiple groups of axons/Schwann cells that were fragmented around and between the multiple thin-films. Cables in the 3-film scaffolds also contained less defined regions of axons/Schwann cells and fibroblasts.

Near the intact proximal nerve stump, the axons were grouped into a large circular cross-section. At further distances into the scaffold, the regeneration cable consists of a more centralized core of regenerating axons (and co-localized Schwann cells). The core of regenerating axons becomes increasingly ellipsoidal, flattening out to conform to the surface of the oriented thin-film that spans the centerline of the scaffold. At distances past the scaffold midpoint, the regeneration cable began to spread out again, and gradually regained a circular cross-section as it approached the distal nerve stump. The regeneration cable was also shaped by the thin-film, although there was a secondary influence in many of the scaffolds: while the interior walls of the scaffold were smooth, the junctions where the scaffold was cut and glued during the fabrication process provided a rough substrate allowing cellular attachment. As a result the regeneration cables in many of the scaffolds had a rectangular cross-section. The grouping of regenerating axons were into mini-fascicles. The distribution of these axonal groupings was relatively uniform within the core of regenerating axons.

The formation of the regenerating axons and surrounding regeneration cable was influenced by the placement of the oriented thin-films. In the 3-film case, however, the regeneration cable was typically larger in size, surrounding all 3 thin-films distributed within the scaffold. The core of axons and Schwann cells, was fragmented into discrete sections that were centered around one or more of the thin-films, often in a non-symmetric fashion. Sparsely distributed axons were more frequently observed within the 3-film scaffolds, particularly in areas between the two outer thin-films. The distribution of axons showed an area of densely grouped axons below the bottom thin-film, but the axons in between the thin-films were more sparsely distributed. Few axons were located above the outer thin-film in this scaffold.

When comparing the 6 wk and 13 wk time points for a given scaffold type, the observed patterns of regeneration were seen to be similar, though regeneration at the 13 week time point was clearly more advanced. For example, the regeneration cables at the later time point were comparatively larger and more developed, especially toward the distal end of the scaffold. There were also differences between the two time points in the cellular make-up of the regeneration cable. Most visibly, while axons were always observed to be co-localized with Schwann cells at both time points, distal portions of the regeneration cables in the 6 wk scaffolds were occupied by Schwann cells alone.

The 3-film scaffolds also resulted in a higher distribution of sparsely scattered axons, not part of a dense cable and lacking any form of fascicular arrangement. This disorganized growth was especially apparent within areas between the two outer thin-films. Areas of scattered and disorganized growth were characterized by the increased presence of vimentin+fibroblasts.

Several scaffolds were sectioned longitudinally to give a different perspective of regeneration. While not designed to examine the precise time course of regeneration cable formation, it is worth noting the observations of the scaffolds sectioned longitudinally at the 2 wk time point. The axons in both scaffold types migrated approximately ⅓ of the way through the scaffolds. The thin-films resulted in some compartmentalization of the scaffold with some degree of cellular segregation. Some compartments were preferentially occupied compared to others, based partially on the positioning of the sutured nerve stump within the scaffold.

From both the 1-film and 3-film groups at the 6 week time point, one scaffold was set aside for longitudinal, as opposed to cross-sectional, cryosectioning. Four animals from each group were left for cross-sectional cryosectioning and quantitative assessment.

The scaffolds were encased in a thin envelope of fibrous tissue, as is characteristic of implanted foreign objects. The visible inflammatory response to the implanted scaffolds was otherwise minimal.

Stains for migrated fibroblasts and macrophages revealed a minimal inflammatory response. ED-1+macrophages could be seen in a thin sparse layer on the interior and exterior surfaces of the guidance channel walls, and scattered within the channel, mainly around the periphery of the regeneration cables. Vimentin staining was used to visualize fibroblasts in the scaffold cross-sections. A thick layer of circumferentially aligned fibroblasts were present surrounding the outer channel walls, and a similar formation was found in a band on the periphery of the regeneration cable, whose interior consisted of a distinct region of co-localized axons and Schwann cells. Though vimentin is known to also stain Schwann cells, double staining with S-100 marked the inner Schwann cell rich region as clearly distinct from the surrounding fibroblast rich region.

As a measure of functional regeneration, the RGMW was calculated for each animal. A relative increase in the mass of denervated gastrocnemius muscles indicates a reversal in atrophy, and can thus be used to assess function reinnervation. The average relative muscle weights were not significantly different between the two groups (t-test: p=0.3).

As another further measure of anatomical and functional reinnervation, the gastrocnemius muscles were cryosectioned after weighing and immunostained to reveal motor endplates, regenerated axons, and synaptic vesicles 2 protein, which is found in functional synaptic terminals. The co-localization of these cellular components indicated an innervated and functional motor endplate, and then the percentage of innervated to deinnervated motor endplates was used as a measure of reinnervation.

In the healthy contralateral gastrocnemius muscles, used as controls, the percentage of reinnervated motor endplates was close to the near 100% that would be expected for normal muscle. In the muscles from the operated limbs, percentages were much lower for both scaffold types.

All 13 week time point animals from the 1-film and 3-film groups underwent electrophysiological assessment at the end of their regeneration times, in order to compare function regeneration through each scaffold type. Nerve conduction velocity (NCV) through the regenerated nerve was evaluated for each animal using two pairs of hook electrodes. One electrode pair was used to evoke a series of compound action potentials (CAPs), and the latency until CAP onset as recorded by a second electrode pair was divided by the separation distance between the two electrode pairs in order to calculate the NCV.

Nerves regenerated through 1-film scaffolds demonstrated significantly higher average NCVs as compared to nerves regenerated through the 3-film scaffolds. NCVs through both scaffold types were much lower than in normal, healthy nerves.

In all animals from the 13 week time point, EMG signals, elicited by upstream stimulation of the regenerated nerve, were recorded from the lateral and medial gastrocnemius muscles (LG and MG), the soleus muscle (SOL), and the tibialis anterior muscle (TA). LG, MG, and SOL muscles, which are normally innervated by the tibial nerve, all contracted visibly in response to nerve stimulation, and produced measurable EMG signals. By contrast, the TA muscle, normally innervated by the common peroneal nerve branch, exhibited no visible contractions, and the electrode measuring its EMG signal recorded only a small residual signal matching those from surrounding muscles. When the regenerating tibial nerve was crushed just distal to the stimulating electrode near the end of the electrophysiological measurements, EMG recording from all muscles disappeared. Gastrocnemius muscles reinnervated by nerves regeneration through the 1-layered scaffolds produced significantly higher tetanic force.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these combinations may not be explicitly disclosed, each is specifically contemplated and described herein. For example, if a particular modification of substrate is disclosed and discussed and a number of modifications that can be made to the substrate are discussed, each and every combination and permutation of the substrate are specifically contemplated unless specifically indicated to the contrary. Likewise, any subset or combination of these is also specifically contemplated and disclosed. Similarly, where methods are disclosed to contain specific steps, combinations or subsets of these steps are contemplated herein.

Optional or optionally means that the subsequently described event or circumstance can, but may not occur, and that the description includes instances where said event or circumstance occurs and instances where it does not.

Unless otherwise expressly stated, it is in no way intended that any method set forth herein be construed as requiring that its steps be performed in a specific order. This holds for any possible non-express basis for interpretation, including: matters of logic with respect to arrangement of steps or operational flow; plain meaning derived from grammatical organization or punctuation; and the number or type of embodiments described in the specification.

1. An implantable device for communicating with biological tissue, comprising:
(a) a substrate having a substrate surface, wherein the substrate is configured to provide cues for directing tissue growth along the substrate surface; and
(b) one or more interfacing units positioned relative to the substrate surface such that the tissue grown along the substrate surface is directed with the cues into operative communication with at least one interfacing unit.

2-68. (canceled)

69. The implantable device of claim 1, wherein one or more of the cues are topographical cues.

70. The implantable device of claim 69, wherein the topographical cue is a physical cue or a biochemical cue.

71. The implantable device of claim 1, wherein at least one interfacing unit is selected from the group consisting of electrodes, optical sensors, optical transmitters, chemical sensors, chemical transmitters, mechanical sensors, mechanical stimulators, thermal sensors, thermal transmitters, light transmitters, light receivers, magnetic transmitters, magnetic receivers, fluid transmitters, and fluid receivers.

72. The implantable device of claim 1, wherein at least one interfacing unit is an electrode and wherein neural tissue is grown along the surface and directed into operative communication with the electrode.

73. The implantable device of claim 72, wherein the neural tissue is regenerating or regenerated neural tissue.

74. The implantable device of claim 72, wherein the electrode is configured to functionally activate the neural tissue directed into operative communication with the electrode by causing an action potential in an axon or subset of axons of the neural tissue.

75. The implantable device of claim 74, wherein the electrode is further configured to receive electrical signals from the neural tissue directed into operative communication with the electrode.

76. The implantable device of claim 72, wherein the electrode is configured to receive signals from the neural tissue directed into operative communication with the electrode.

77. The implantable device of claim 74, further comprising a second electrode configured to receive electrical signals from the neural tissue directed into operative communication with the second electrode.

78. The implantable device of claim 76, further comprising a second electrode configured to functionally activate the neural tissue directed into operative communication with the second electrode by causing an action potential in an axon or subset of axons of the neural tissue.

79. The implantable device of claim 1, wherein the substrate comprises a plurality of uniaxially oriented fibers made of at least one synthetic or natural polymer.

80. The implantable device of claim 79, wherein the fibers are nanofibers.

81. The implantable device of claim 1, wherein the substrate has a longitudinal axis and the surface is substantially planar.

82. An implantable device for communicating with biological tissue, comprising:
(a) a substrate having a substrate surface, wherein the substrate is configured to topographically direct biological tissue growth along the substrate surface; and
(b) a plurality of electrodes positioned relative to the substrate surface such that tissue grown along the surface is directed into operative communication with one or more of the electrodes.

83. The implantable device of claim 82, wherein the tissue grown along the surface and topographically directed into operative communication with the electrodes is neural tissue.

84. The implantable device of claim 83, wherein one or more electrodes are configured to functionally activate the neural tissue topographically directed into operative communication with the electrodes by causing an action potential in an axon or subset of axons of the neural tissue.

85. The implantable device of claim 84, wherein one or more electrode are further configured to receive electrical signals from the neural tissue topographically directed into operative communication with the electrode.

86. The implantable device of claim 83, wherein one or more electrodes is configured to receive electrical signals from the neural tissue topographically directed into operative communication with the electrode.

87. A method for communicating with biological tissue in a subject, comprising:
(a) positioning an implantable device within the subject, wherein the implantable device comprises a substrate having a substrate surface and an interfacing unit;
(b) allowing tissue to grow along the substrate surface, wherein the substrate provides cues that direct the tissue growing along the substrate surface into operative communication with the interfacing unit; and
(c) communicating with the tissue by receiving signals from the tissue through the interfacing unit or by directing signals into the tissue through the interfacing unit.
